(19) United States
(12) Patent Application Publication
(10) Pub. No.: US 2025/0376729 A1
(43) Pub. Date: Dec. 11, 2025

(54) MIRNA-BASED BIOMARKER FOR MUSCLE WASTING IN OSTEOPOROTIC PATIENTS

(71) Applicant: Ospedale Galeazzi S.P.A., Milano (IT)

(72) Inventors: Martina Faraldi, Milano (IT); Giovanni Lombardi, Milano (IT); Veronica Sansoni, Milano (IT); Silvia Perego, Milano (IT); Marta Sofia Gomarasca, Milano (IT); Sabrina Luigia Corbetta, Milano (IT)

(73) Assignee: Ospedale Galeazzi S.P.A., Milano (IT)

(21) Appl. No.: 19/127,806

(22) PCT Filed: Nov. 7, 2023
(86) PCT No.: PCT/EP2023/081054

§ 371 Date: May 7, 2025

(30) Foreign Application Priority Data
Nov. 8, 2022 (IT) 102022000023046

(51) Int. Cl. C12Q 1/6883

(52) U.S. Cl. CPC C12Q 1/6883; C12Q 2600/112; C12Q 2600/178

(57) ABSTRACT

It forms an object of the present invention a method for the in vitro diagnosis, prognosis and/or treatment monitoring of osteosarcopenia, wherein the method comprises the steps: a) making available a test sample from the subject; b) collecting the microRNAs (miRNAs) contained in the test sample; c) determining the expression profile of a predetermined set of miRNA; d) comparing said expression profile to one or several reference expressions profiles, wherein the comparison of said determined expression profile to said one or several reference expressions profiles allows for the diagnosis, prognosis and/or treatment monitoring of the disease.

### BACKGROUND

Osteoporosis and sarcopenia are disorders predominantly occurring in elderly people: osteoporosis is characterized by the decrease of bone mineral density (BMD), the increase of bone fragility and fracture risk, while sarcopenia is characterized by the decrease of muscle mass, muscle function, and the increase of risk of fall [1,2]. Osteoporosis and sarcopenia often coexist in an increasingly discussed syndrome called osteosarcopenia, leading to significantly worsened outcomes then those observed in one of the two pathologies alone [3].

Osteoporosis affects millions of people worldwide and, only in Europe, since 2010, 22 million women and 5.5 million men were osteoporotic, and the number of fractures was estimated at 3.5 million. Osteoporotic fractures carry an economic burden of €37 billion in 2010 that was predicted to increase by 25% in 2025, in the EU [4].

Determining the prevalence rate for sarcopenia is difficult due to the lack of a standard definition for sarcopenia [2,5,6]. Since 2010, sarcopenia has affected >50 million of people worldwide, estimated to rise to >200 million over the next 40 years, with an increasing impact on public health burden [7].

Until now, a unique and standardized diagnostic method for osteosarcopenia has not been introduced into clinical practice. Osteosarcopenia diagnosis is indeed based on the combination of osteoporosis and sarcopenia criteria. Dual-energy X-ray absorptiometry (DXA) is recommended for BMD measurement: the World Health Organization has defined as osteoporotic those people who show a T-score of BMD lower than −2.5 [8].

Sarcopenia diagnosis requires the combination of different methods that measure skeletal muscle mass and function: DXA and bioelectrical impedance analysis (BIA) are used for skeletal muscle mass measurement, while grip strength and gait speed for skeletal muscle mass function and strength assessment [5,9]. For both osteoporosis and sarcopenia, tools to evaluate the risk of pathologic symptoms, such as FRAX (prediction of the 10-year risk of hip fractures) and SARC-F (five-item questionnaire to evaluate sarcopenia risk) [11], have been validated into clinical practice, while no rapid test or screening to predict osteosarcopenia or to assess any risk associated with the pathology exist.

Therefore, predictive and diagnostic tools to manage, and eventually to prevent, osteosarcopenia should be implemented into clinical practice.

The assessment of bone and skeletal muscle metabolism through the measurement of circulating biomarkers as C-terminal cross-linked telopeptide (CTX), osteocalcin (OC), tartrate-resistant acid phosphatase (TRAP), bone alkaline phosphatase (BALP), insulin-like growth factor (IGF-1) have been suggested to improve osteosarcopenia early diagnosis [12,13].

Circulating microRNAs (miRNAs) represent the most promising circulating molecules to this purpose. miRNAs are small noncoding RNAs involved in the post-transcriptional regulation of gene expression. miRNAs are synthetized by almost all tissue and have been demonstrated to play key roles in multiple biological process, including bone and skeletal muscle homeostasis [14-16]. miRNAs can also be secreted, in response to both physiological and pathological stimuli, into body fluids in stable forms, associated with proteins or encapsulated into extracellular vesicles [17]. Being stable, easily detectable, and sensitive to changes in physiological status, circulating miRNAs potentially possess the entire set of features that makes a non-invasive biomarker relevant in monitoring pathophysiological processes, with diagnostic and prognostic potential [17].

It has been demonstrated that alterations in expression or circulating levels of specific miRNAs have been associated to the development and progression of skeletal muscle or bone diseases that occur during aging, as sarcopenia and osteoporosis [18-25]. However, no evidence exist on circulating miRNA signatures associated with an osteosarcopenic status, that identifies the coexistence of both skeletal muscle mass and BMD decrease in elderly.

In this contest, this study aims at identifying a unique and innovative method, based on plasma miRNA measurement, to describe skeletal muscle mass status in postmenopausal osteoporotic women.

### DESCRIPTION

The authors of the present invention have surprisingly demonstrated a strong and specific correlation between miRNA content and osteosarcopenia.

The authors have surprisingly demonstrated that specific patterns of miRNAs are activated under detrimental conditions.

In an embodiment, it is here described a pathology specific miRNA profile within miRNA from biological fluids for early diagnosis, prognosis and/or treatment monitoring.

### DRAWINGS DESCRIPTION

FIG. 1: circulating level of miRNAs 1.5-fold (a) up and (b) down-regulated (1^{st} tertile vs. 3^{rd} tertile).

### DEFINITIONS

A “miRNA” is a naturally occurring, small non-coding RNA that is about 17 to about 25 nucleotides (nt) in length in its biologically active form that negatively regulates mRNA translation on a sequence-specific manner. Identified miRNAs are registered in the miRNA database miRBase (http://microma.sanger.ac.uk/).

A “sample”, as defined herein, is a small part of a subject, representative of the whole and may be constituted by a body fluid sample. Body fluid samples may be blood, plasma, serum, urine, sputum, cerebrospinal fluid, milk, or ductal fluid samples and may likewise be fresh, frozen, or fixed. Samples may be removed surgically, by extraction i.e., by hypodermic or other types of needles, by microdissection or laser capture. The sample should contain any biological material suitable for detecting the desired biomarkers (miRNAs), thus, said sample should advantageously comprise cell material from the subject.

A “reference sample”, as used herein, means a sample obtained from individuals, preferably two or more individuals, known to be free of sarcopenia. The suitable reference expression levels of miRNAs can be determined by measuring the expression levels of said miRNAs in several suitable individuals, and such reference levels can be adjusted to specific populations. In a preferred embodiment, the reference sample is obtained from a pool of healthy individuals. The expression profile of the miRNAs in the reference sample can, preferably, be generated from a population of two or more individuals; for example, the population can comprise 3, 4, 5, 10, 15, 20, 30, 40, 50 or more subjects.

An “individual”, as used herein, refers to a mammal, human or non-human, under observation, preferably a human being, preferably a woman.

As used herein, the expression “diagnosis” or “diagnosing” relates to methods by which the skilled person can estimate and even determine whether an individual is suffering from a given disease or condition.

Along with diagnosis, clinical disease prognosis is also an area of great concern and interest. It is important to know the stage and rapidity of advancement of the disease to plan the most effective therapy. If a more accurate prognosis can be made, appropriate therapy, and in some instances less severe therapy for the patient can be chosen.

Further, the expression “method of diagnosing” as used herein relates to a method that may essentially consist of the steps mentioned below or may include additional steps. However, it must be understood that the method, in a preferred embodiment, is a method that is carried out in vitro, i.e., it is not carried out in the human or animal body.

In an embodiment, it is here described a method for the in vitro diagnosis, prognosis and/or treatment monitoring of osteosarcopenia in a subject, wherein the method comprises the steps of:
a) making available a test sample from the subject;
b) collecting the microRNAs (miRNAs) contained in the test sample
c) determining the expression profile of a predetermined set of miRNAs;
d) comparing said expression profile to one or several reference expressions profiles,
wherein the comparison of said determined expression profile to said one or several reference expression profiles allows for the diagnosis, prognosis and/or treatment monitoring of the disease.

The expression levels of a plurality of miRNAs are determined as expression level values and, in a further preferred embodiment, said comparison step d) comprises mathematically combining the expression level values of said plurality miRNAs by applying an algorithm to obtain a normalized expression level relative to at least one reference pattern of expression levels.

In a preferred embodiment, the determination of the expression profile in said step c) is obtained by the use of a method selected from the group consisting of a Sequencing-based method, an array-based method and a PCR based method.

In an embodiment, said test sample is plasma from venous blood.

In an embodiment, said method further comprises treating said individual with a suitable therapy based on the diagnosis of said individual.

In an embodiment, it is here described a method for the in vitro diagnosis, prognosis and/or treatment monitoring of osteosarcopenia in a subject, wherein the method comprises the steps of:
a) Measuring the expression level of at least one nucleic acid in a test sample from the subject;
b) Receiving the expression level of the at least one nucleic acid in the test sample by a computer and;
c) Comparing the expression level of the at least one nucleic acid in the test sample to a level in a base sample for the same at least one nucleic acid, and
d) Receiving a result comparing the expression levels of the at least one nucleic acid in the test sample measured in a) and the base sample measured in c),
e) Diagnosis or determining the prognosis of osteosarcopenia based on altered expression of the at least one nucleic acid in the test sample as compared to the base sample as determined in a computer, and
f) Treating the subject for osteosarcopenia based on the diagnosis or prognosis, wherein the at least one nucleic acid is a miRNA.

In an embodiment, said expression profile is determined of miRNAs selected from the group consisting of hsa-let-7a-5p, hsa-let-7b-3p, hsa-let-7b-5p, hsa-let-7c-5p, hsa-let-7d-3p, hsa-let-7d-5p, hsa-let-7e-5p, hsa-let-7f-5p, hsa-let-7g-5p, hsa-let-7i-5p, hsa-miR-1-3p, hsa-miR-100-5p, hsa-miR-101-3p, hsa-miR-103a-3p, hsa-miR-106a-5p, hsa-miR-106b-3p, hsa-miR-106b-5p, hsa-miR-107, hsa-miR-10b-5p, hsa-miR-122-5p, hsa-miR-125a-5p, hsa-miR-125b-5p, hsa-miR-1260a, hsa-miR-126-3p, hsa-miR-126-5p, hsa-miR-127-3p, hsa-miR-128-3p, hsa-miR-130a-3p, hsa-miR-130b-3p, hsa-miR-132-3p, hsa-miR-133a-3p, hsa-miR-133b, hsa-miR-136-3p, hsa-miR-136-5p, hsa-miR-139-5p, hsa-miR-140-3p, hsa-miR-140-5p, hsa-miR-141-3p, hsa-miR-142-3p, hsa-miR-142-5p, hsa-miR-143-3p, hsa-miR-144-3p, hsa-miR-144-5p, hsa-miR-145-5p, hsa-miR-146a-5p, hsa-miR-146b-5p, hsa-miR-148a-3p, hsa-miR-148b-3p, hsa-miR-150-5p, hsa-miR-151a-3p, hsa-miR-151a-5p, hsa-miR-152-3p, hsa-miR-154-5p, hsa-miR-155-5p, hsa-miR-15a-5p, hsa-miR-15b-3p, hsa-miR-15b-5p, hsa-miR-16-2-3p, hsa-miR-16-5p, hsa-miR-17-5p, hsa-miR-181a-5p, hsa-miR-185-5p, hsa-miR-186-5p, hsa-miR-18a-5p, hsa-miR-18b-5p, hsa-miR-191-5p, hsa-miR-192-5p, hsa-miR-193a-5p, hsa-miR-194-5p, hsa-miR-195-5p, hsa-miR-197-3p, hsa-miR-199a-3p, hsa-miR-199a-5p, hsa-miR-19a-3p, hsa-miR-19b-3p, hsa-miR-200a-3p, hsa-miR-200c-3p, hsa-miR-205-5p, hsa-miR-208a-3p, hsa-miR-20a-5p, hsa-miR-20b-5p, hsa-miR-210-3p, hsa-miR-2110, hsa-miR-215-5p, hsa-miR-21-5p, hsa-miR-221-3p, hsa-miR-222-3p, hsa-miR-223-3p, hsa-miR-223-5p, hsa-miR-22-3p, hsa-miR-22-5p, hsa-miR-23a-3p, hsa-miR-23b-3p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-29a-3p, hsa-miR-29b-3p, hsa-miR-29c-3p, hsa-miR-301a-3p, hsa-miR-30a-5p, hsa-miR-30b-5p, hsa-miR-30c-5p, hsa-miR-30d-5p, hsa-miR-30e-3p, hsa-miR-30e-5p, hsa-miR-320a, hsa-miR-320b, hsa-miR-320c, hsa-miR-320d, hsa-miR-324-3p, hsa-miR-324-5p, hsa-miR-32-5p, hsa-miR-326, hsa-miR-328-3p, hsa-miR-331-3p, hsa-miR-335-3p, hsa-miR-335-5p, hsa-miR-338-3p, hsa-miR-339-3p, hsa-miR-339-5p, hsa-miR-33a-5p, hsa-miR-342-3p, hsa-miR-34a-5p, hsa-miR-361-5p, hsa-miR-362-3p, hsa-miR-363-3p, hsa-miR-365a-3p, hsa-miR-374a-5p, hsa-miR-374b-5p, hsa-miR-375, hsa-miR-376a-3p, hsa-miR-376c-3p, hsa-miR-378a-3p, hsa-miR-382-5p, hsa-miR-409-3p, hsa-miR-421, hsa-miR-423-3p, hsa-miR-423-5p, hsa-miR-424-5p, hsa-miR-425-3p, hsa-miR-425-5p, hsa-miR-451a, hsa-miR-454-3p, hsa-miR-483-5p, hsa-miR-484, hsa-miR-485-3p, hsa-miR-486-5p, hsa-miR-495-3p, hsa-miR-497-5p, hsa-miR-501-3p, hsa-miR-502-3p, hsa-miR-505-3p, hsa-miR-532-3p, hsa-miR-532-5p, hsa-miR-543, hsa-miR-574-3p, hsa-miR-584-5p, hsa-miR-590-5p, hsa-miR-629-5p, hsa-miR-652-3p, hsa-miR-660-5p, hsa-miR-7-1-3p, hsa-miR-766-3p, hsa-miR-874-3p, hsa-miR-877-5p, hsa-miR-885-5p, hsa-miR-92a-3p, hsa-miR-92b-3p, hsa-miR-93-3p, hsa-miR-93-5p, hsa-miR-99a-5p, hsa-miR-99b-5p.

In an embodiment, a pattern of at least 2 down-regulated specific miRNAs listed above is an indicator of osteosarcopenia. Preferably, said 2 down-regulated miRNA are hsa-miR-146a-5p (SEQ ID NO: 45) and hsa-miR-126-5p (SEQ ID NO: 25).

In an embodiment, a pattern of at least 3 down-regulated specific miRNAs listed above is an indicator of osteosarcopenia. Preferably, said pattern is the pattern listed in Table 1.

TABLE 1
osteosarcopenia, miRNA pattern

| miRNA | |
|---|---|
| hsa-miR-146a-5p (SEQ ID NO: 45) | − |
| hsa-miR-126-5p (SEQ ID NO: 25) | − |
| hsa-miR-425-5p (SEQ ID NO: 147) | − |

(+, upregulated; −, downregulated)

In a preferred embodiment, said pattern is the pattern listed in Table 2.

TABLE 2
osteosarcopenia, preferred miRNA pattern

| miRNA | |
|---|---|
| hsa-miR-146a-5p (SEQ ID NO: 45) | − |
| hsa-miR-25-3p (SEQ ID NO: 95) | + |
| hsa-miR-126-5p (SEQ ID NO: 25) | − |
| hsa-miR-145-5p (SEQ ID NO: 44) | + |
| hsa-miR-425-5p (SEQ ID NO: 147) | − |

(+, upregulated; −, downregulated)

In an embodiment, combination of hsa-miR-146a-5p (SEQ ID NO: 45), hsa-miR-126-5p (SEQ ID NO: 25), hsa-miR-425-5p (SEQ ID NO: 147) display an AUC of 0.914.

In an embodiment, combination of hsa-miR-146a-5p (SEQ ID NO: 45), hsa-miR-126-5p (SEQ ID NO: 25), has-145-5p (SEQ ID NO: 44), has-miR-25-3p (SEQ ID NO: 95) display an AUC of 0.901 respectively.

The AUC of miRNAs combination was calculated on the average value of considered miRNAs.

In an embodiment, detecting the level of the at least one miRNA comprises: (i) generating a first strand cDNA for each miRNA within the sample by performing a reverse transcription assay using a primer that specifically binds the at least one miRNA or using a universal primer, thereby producing cDNA; (ii) amplifying the produced cDNA with specific primers for at least one miRNA, wherein the SYBR green dye emits a fluorescent signal upon binding to all double strand cDNA; and (iii) using a detector to detect the signal emitted by the fluorescent dye.

In an embodiment, detecting the level of the at least one miRNA is by quantitative PCR.

In an embodiment, detecting the level of the at least one miRNA further comprises performing a reverse transcription reaction on the at least one miRNA using at least one primer or probe specific for the at least one miRNA or using at least one universal primer.

In an embodiment, the present invention relates to a method for early diagnosis of osteosarcopenia, where said method comprises the quantitative-qualitative measurement of the miRNA selected from the group listed above.

In a further aspect, a kit for diagnosis and prognosis of osteosarcopenia is described, comprising:
a) means for determining the miRNA expression profile of a miRNA sample of plasma of a subject, and
b) at least one reference set of miRNA profile characteristic for a particular condition.

### EXAMPLES

### Methods

### Study Population

28 postmenopausal osteoporotic women (61-85 years old) were recruited. All participants were characterized for weight (kg), height (m), BMI (kg/m²), total fat mass (kg) determined by MRI, and ASMMI, Appendicular Skeletal Muscle Mass Index (kg/m²), and BMD, bone mineral density (T-score), measured by DXA.

To highlight the differences of osteoporotic women with different muscle mass, the overall population was divided in tertiles, based on ASMMI (Kg/m²) (1^{st} tertile N=9; 2^{nd} tertile N=10; 3^{rd} tertile N=9) (Table 2): the 1^{st} tertile included osteoporotic women with low skeletal muscle mass while the 3^{rd} tertile include osteoporotic women with normal skeletal muscle mass.

### Sample Collection

Venous blood was collected in spray-coated dipotassium ethylendiaminotetraacetate (K2EDTA) (tubes (BD Vacutainer®, Becton Dickinson, Milano, Italia). Blood samples were homogenized for 15 min, at room temperature (RT), and then centrifuged at 2000 g for 10′ to get plasma. Plasma aliquots were immediately frozen at −80° C. until processing.

miRNA Profiling

Before processing, thawed plasma was centrifuged 5 min at 3000 g. miRNA-enriched total RNA was extracted from each sample and treated with DNase according to miRCURY™ RNA Isolation Kit protocol (Exiqon A/S) and stored at −80° C. miRNA-enriched total RNA was reverse transcribed with miRCURY LNAT Universal cDNA synthesis kit Il and stored at −20° C. until assayed. The spike-in UniSp2, UniSp4, UniSp5 (Exiqon) were added to each sample at the recommended concentration of 2.0 fmol/μL, 2.0.10-2 fmol/μL, 2.0·10-4 fmol/μL, to check the efficiency of RNA extraction, while the spike-in UniSp6 and cel-39-3p (Exiqon) (1.5·10-1 fmol/μL, and 2.0·10-3 fmol/μL respectively), to check the efficiency of reverse transcription.

miRNA expression profile was performed through quantitative real time polymerase chain reaction (qPCR) using serum/plasma miRCURY LNA™ miRNA focus panel (Exiqon A/S), containing 179 LNA™ primer set for the most relevant circulating miRNAs, 5 RNA spike-in control primer sets, 2 blank wells, and 6 inter-plate calibrators. qPCR was carried out on a StepOne Plus instrument (Applied Biosystem, Foster City, CA, USA), using ExiLENT SYBR Green 2× Master Mix (Exiqon). Polymerase activation for 10 min at 95° C. was followed by 40×10 s-amplification cycles at 95° C., 60 s at 60° C., and melting curves. GenEx software ver6 (Exiqon) was used to perform qPCR data analysis. The quantification cycle (Cq) of the inter-plate calibrator (IPC) was used to adjust the miRNA Cq values from the RT-qPCR plate runs of each sample. Only miRNAs with an adjusted Cq<37 were considered for further analysis. The relative expression of analyzed miRNAs was calculated by the 2-ΔΔCq method, normalizing on global mean. Hemolysis was checked by the hsa-miR-23a and hsa-miR-451a Cq difference (positive if >7).

miRNA profile analysis was performed dividing the population in tertiles based on ASMMI, comparing miRNA expression level between first tertile and third tertile. Only miRNAs significantly ≥1.5-fold either up- or down-regulated were considered.

### Statistical Analysis

Differences of T-score, ASMMI, and skeletal muscle mass among the three tertiles were analyzed by ordinary Kruskal-Wallis test with Dunn's multiple comparison test. miRNA expression level between osteoporotic women of the 1^{st} tertile and of the 3^{rd} tertile were compared through non-parametric Mann-Withney test. miRNA diagnostic value was analyzed through receiver operating characteristic (ROC) curves. ROC curve related area under curve (AUC), sensitivity, and sensibility was also calculated [27]. Univariate and multivariate regression models were applied to study the association of miRNAs with clinical parameters, considering all population (1^{st}, 2^{nd}, and 3^{rd} tertile). Results were considered statistically significant if p-values <0.05. These statistical analyses were performed with Prism® v6.01 (GraphPad Software Inc., La Jolla, CA, USA). Univariate and multivariate analysis were performed on R 64 3.5.2.

### Results

### Example 1: Characterization of Osteoporotic Women Population

Details on the characterization of all the participants are shown in Table 3. Considering the whole population divided in tertiles based on ASMMI (kg/m²), the three groups significantly differ for skeletal muscle mass (kg) (<0.001), for ASMMI (kg/m²) (<0.001), while no differences were observed for t-score (0.521) (Table 4).

For miRNAs analyses, osteoporotic women included in 1^{st} tertile (osteoporotic women with low skeletal muscle mass) and 3^{rd} tertile (osteoporotic women with normal skeletal muscle mass) were considered.

TABLE 3
Population characteristics
Patients n = 28
Sex

| Men | / |
|---|---|
| Women | 28 |

Age (years)

| | |
|---|---|
| (range) | 61.80-85.40 |

Weight (kg)

| | |
|---|---|
| (avg ± SD) | 55.58 ± 8.03 |

Height (m)

| | |
|---|---|
| (avg ± SD) | 1.54 ± 0.05 |

BMI (kg/m²)

| | |
|---|---|
| (avg ± SD) | 23.62 ± 4.05 |

ASMMI (kg/m²)

| | |
|---|---|
| <5.5 | 11 |
| >5.5 | 17 |

Total fat (kg)

| | |
|---|---|
| (avg ± SD) | 20.73 ± 5.72 |

T-score

| | |
|---|---|
| (avg ± SD) | −2.75 ± 0.23 |
| (range) | [−2.50-3.10] |

TABLE 4
Tertile characteristics

| | 1^{st} (n = 9) | 2^{nd} (n = 10) | 3^{rd} (n = 9) | |
|---|---|---|---|---|
| | avg ± SD | | | p-value |
| t-score | −2.82 ± 0.24 | −2.72 ± 0.23 | −2.70 ± 0.22 | 0.521 |
| ASMMI | 4.88 ± 0.40 | 5.73 ± 0.23 | 6.40 ± 0.22 | <0.001 |
| (kg/m²) | | | | |
| Muscle | 11.83 ± 1.203 | 13.88 ± 1.64 | 14.59 ± 0.94 | <0.001 |
| mass (kg) | | | | |

### Example 2: MiRNA Profiling and Selection

The circulating level of 179 miRNAs was analyzed in plasma of 18 osteoporotic women with different skeletal muscle mass (1^{st} tertile N=9; 3^{rd} tertile N=9). The fold change and the respective p-value of all miRNAs are shown in Table 5.

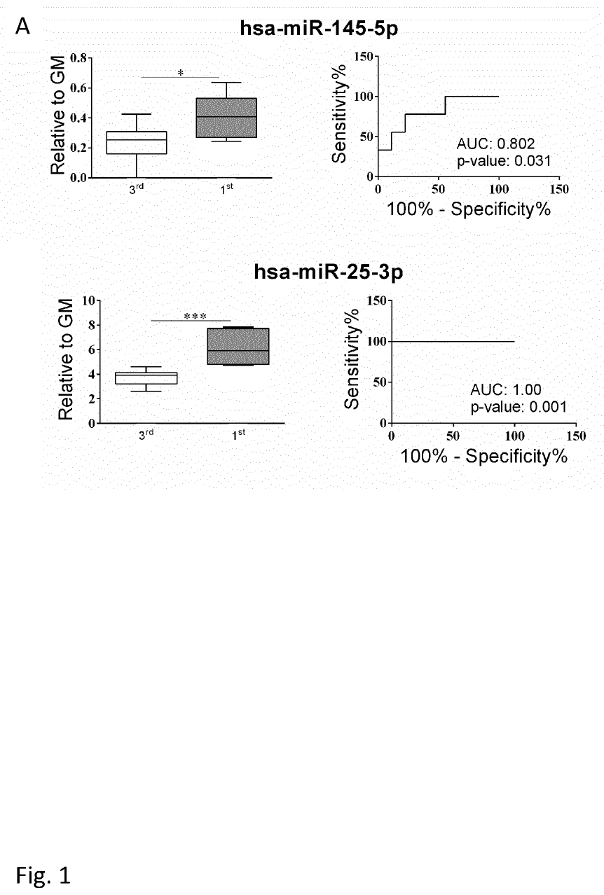

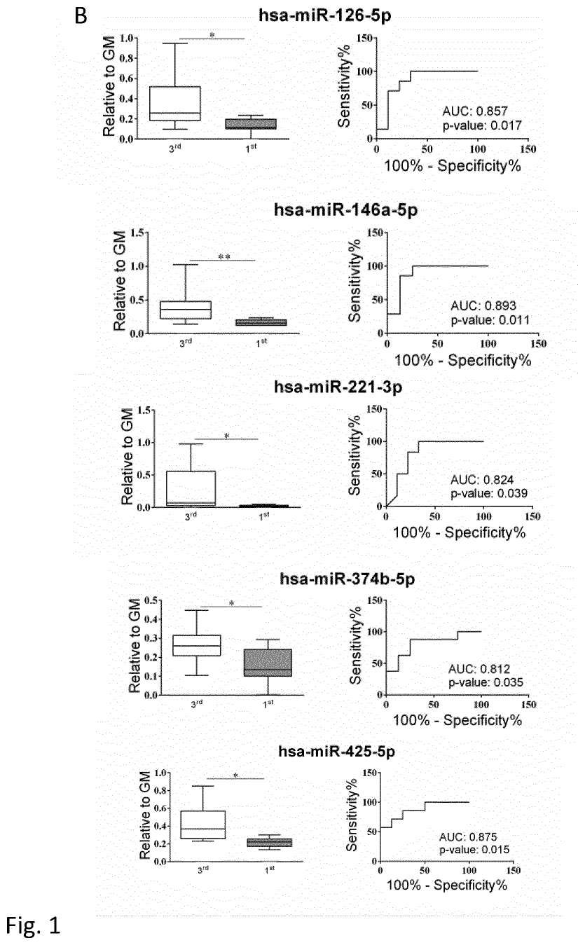

Of the 179 analyzed miRNAs, 2 miRNAs (hsa-miR-145-5p SEQ ID NO: 44, hsa-miR-25-3p SEQ ID NO: 95) were significantly 1.5-fold up-regulated, while 5 (hsa-miR-126-5p SEQ ID NO: 25, hsa-miR-146a-5p SEQ ID NO: 45, hsa-miR-221-3p SEQ ID NO: 86, hsa-miR-374b-5p SEQ ID NO: 135, and hsa-miR-425-5p SEQ ID NO: 147) were significantly 1.5 fold down-regulated in the 1^{st} tertile compared to the 3^{rd} tertile (FIG. 1).

The same population of osteoporotic women was used to define miRNAs' diagnostic accuracy.

ROC curve analysis was conducted on the 7 plasma miRNAs indicated above, to assess their sensitivity and specificity in discriminating between osteoporotic women with low skeletal muscle mass and osteoporotic women with normal skeletal muscle mass. The AUC measured for the 7 miRNAs, singularly, range from 0.802 and 1.000 (p-value <0.050) as shown in FIG. 1 and Table 6. Based on the Youden's index, sensitivity and specificity of the 7 identified miRNAs are 100 and 66.67 (hsa-miR-126-5p SEQ ID NO: 25), 77.78 and 77.78 (hsa-miR-145-5p SEQ ID NO: 44), 100.00 and 75.00 (hsa-miR-146a-5p SEQ ID NO: 45), 100.00 and 66.67 (hsa-miR-221-3p SEQ ID NO:86), 87.50 and 100 (hsa-miR-25-3p SEQ ID NO:95), 87.50 and 75.00 (hsa-miR-374b-5p SEQ ID NO:135), 85.71 and 75.00 (hsa-miR-425-5p SEQ ID NO: 147), respectively.

TABLE 5

| | Seq | Nucleotide | | | |
|---|---|---|---|---|---|
| | ID | accession | | Fold | |
| miRNA | No | number | Sequence | change | p-value |
| hsa-let-7a-5p | 1 | LM378749 | UGAGGUAGUAGGUUGUAUAGUU | 0.6677 | 0.2930 |
| hsa-let-7b-3p | 2 | LM380163 | CUAUACAACCUACUGCCUUCCC | 11297 | 0.9528 |
| hsa-let-7b-5p | 3 | LM378750 | UGAGGUAGUAGGUUGUGUGGUU | 11600 | 0.3799 |
| hsa-let-7c-5p | 4 | LM378751 | UGAGGUAGUAGGUUGUAUGGUU | 0.7562 | 0.3481 |
| hsa-let-7d-3p | 5 | LM380164 | CUAUACGACCUGCUGCCUUUCU | 0.5636 | 0.0502 |
| hsa-let-7d-5p | 6 | LM378752 | AGAGGUAGUAGGUUGCAUAGUU | 0.7404 | 0.9758 |
| hsa-let-7e-5p | 7 | LM378753 | UGAGGUAGGAGGUUGUAUAGUU | 0.7868 | 0.4506 |
| hsa-let-7f-5p | 8 | LM378754 | UGAGGUAGUAGAUUGUAUAGUU | 0.9096 | 0.8427 |
| hsa-let-7g-5p | 9 | LM379032 | UGAGGUAGUAGUUUGUACAGUU | 0.7202 | 0.5346 |
| hsa-let-7i-5p | 10 | LM379033 | UGAGGUAGUAGUUUGUGCUGUU | 10762 | 0.9758 |
| hsa-miR-100-5p | 12 | LM378785 | AACCCGUAGAUCCGAACUUGUG | 0.9551 | 0.9307 |
| hsa-miR-101-3p | 13 | LM378786 | UACAGUACUGUGAUAACUGAA | 0.8012 | 0.8427 |
| hsa-miR-103a-3p | 14 | LM378788 | AGCAGCAUUGUACAGGGCUAUGA | 0.8517 | 0.3799 |
| hsa-miR-106a-5p | 15 | LM378790 | AAAAGUGCUUACAGUGCAGGUAG | 11535 | 0.5346 |
| hsa-miR-106b-3p | 16 | LM380336 | CCGCACUGUGGGUACUUGCUGC | 12480 | 0.5625 |
| hsa-miR-106b-5p | 17 | LM379200 | UAAAGUGCUGACAGUGCAGAU | 11444 | 0.7137 |
| hsa-miR-107 | 18 | LM378791 | AGCAGCAUUGUACAGGGCUAUCA | 0.5698 | 0.2672 |
| hsa-miR-10b-5p | 19 | LM378895 | UACCCUGUAGAACCGAAUUUGUG | 17201 | 0.1348 |
| hsa-miR-122-5p | 20 | LM379039 | UGGAGUGUGACAAUGGUGUUUG | 0.9923 | 0.9758 |
| hsa-miR-125a-5p | 21 | LM379061 | UCCCUGAGACCCUUUAACCUGUGA | 0.8277 | 0.7137 |
| hsa-miR-125b-5p | 22 | LM379041 | UCCCUGAGACCCUAACUUGUGA | 19346 | 0.2170 |
| hsa-miR-1260a | 23 | LM380704 | AUCCCACCUCUGCCACCA | 14374 | 0.5346 |
| hsa-miR-126-3p | 24 | LM379063 | UCGUACCGUGAGUAAUAAUGCG | 0.7576 | 0.9758 |
| hsa-miR-126-5p | 25 | LM379062 | CAUUAUUACUUUUGGUACGCG | 0.3479 | 0.0164 |
| hsa-miR-127-3p | 26 | LM379064 | UCGGAUCCGUCUGAGCUUGGCU | 0.7671 | 0.6061 |
| hsa-miR-128-3p | 27 | LM379042 | UCACAGUGAACCGGUCUCUUU | 0.7674 | 0.3676 |
| hsa-miR-130a-3p | 28 | LM379043 | CAGUGCAAUGUUAAAAGGGCAU | 0.8763 | 0.5919 |
| hsa-miR-130b-3p | 29 | LM379211 | CAGUGCAAUGAUGAAAGGGCAU | 11346 | 0.1128 |
| hsa-miR-132-3p | 30 | LM379044 | UAACAGUCUACAGCCAUGGUCG | 12207 | 0.1970 |
| hsa-miR-133a-3p | 31 | LM379045 | UUUGGUCCCCUUCAACCAGCUG | 0.8963 | 0.7967 |
| hsa-miR-133b | 32 | LM379274 | UUUGGUCCCCUUCAACCAGCUA | 0.7075 | 0.5629 |
| hsa-miR-136-3p | 33 | LM380281 | CAUCAUCGUCUCAAAUGAGUCU | 0.0538 | 0.2000 |
| hsa-miR-136-5p | 34 | LM379066 | ACUCCAUUUGUUUUGAUGAUGGA | 0.7121 | 0.4164 |
| hsa-miR-139-5p | 35 | LM378892 | UCUACAGUGCACGUGUCUCCAGU | 0.5873 | 0.7214 |
| hsa-miR-1-3p | 11 | LM379034 | UGGAAUGUAAAGAAGUAUGUAU | 0.6552 | 0.3388 |
| hsa-miR-140-3p | 36 | LM380272 | UACCACAGGGUAGAACCACGG | 13492 | 0.3347 |
| hsa-miR-140-5p | 37 | LM379049 | CAGUGGUUUUACCCUAUGGUAG | 0.6591 | 0.3518 |
| hsa-miR-141-3p | 38 | LM379050 | UAACACUGUCUGGUAAAGAUGG | 0.9113 | 0.9292 |
| hsa-miR-142-3p | 39 | LM379052 | UGUAGUGUUUCCUACUUUAUGGA | 0.7378 | 0.7137 |
| hsa-miR-142-5p | 40 | LM379051 | CAUAAAGUAGAAAGCACUACU | 0.4404 | 0.5052 |
| hsa-miR-143-3p | 41 | LM379053 | UGAGAUGAAGCACUGUAGCUC | 11433 | >0.9999 |
| hsa-miR-144-3p | 42 | LM379054 | UACAGUAUAGAUGAUGUACU | 0.6833 | 0.9090 |
| hsa-miR-144-5p | 43 | LM380275 | GGAUAUCAUCAUAUACUGUAAG | 12332 | 0.8167 |
| hsa-miR-145-5p | 44 | LM379055 | GUCCAGUUUUCCCAGGAAUCCCU | 17160 | 0.0314 |
| hsa-miR-146a-5p | 45 | LM379067 | UGAGAACUGAAUUCCAUGGGUU | 0.5612 | 0.0175 |
| hsa-miR-146b-5p | 46 | LM379768 | UGAGAACUGAAUUCCAUAGGCU | 0.7355 | 0.2069 |
| hsa-miR-148a-3p | 47 | LM378885 | UCAGUGCACUACAGAACUUUGU | 0.9168 | 0.6517 |
| hsa-miR-148b-3p | 48 | LM379264 | UCAGUGCAUCACAGAACUUUGU | 0.7132 | 0.5625 |
| hsa-miR-150-5p | 49 | LM379069 | UCUCCCAACCCUUGUACCAGUG | 0.8316 | 0.6517 |
| hsa-miR-151a-3p | 50 | LM379262 | CUAGACUGAAGCUCCUUGAGG | 0.5946 | 0.1348 |
| hsa-miR-151a-5p | 51 | LM380357 | UCGAGGAGCUCACAGUCUAGU | 0.5797 | 0.0592 |
| hsa-miR-152-3p | 52 | LM379056 | UCAGUGCAUGACAGAACUUGG | 0.9085 | 0.4799 |
| hsa-miR-154-5p | 53 | LM379070 | UAGGUUAUCCGUGUUGCCUUCG | 18403 | 0.2433 |
| hsa-miR-155-5p | 54 | LM379168 | UUAAUGCUAAUCGUGAUAGGGGU | 0.7859 | 0.4638 |
| hsa-miR-15a-5p | 55 | LM378755 | UAGCAGCACAUAAUGGUUUGUG | 0.6048 | 0.5346 |
| hsa-miR-15b-3p | 56 | LM380264 | CGAAUCAUUAUUUGCUGCUCUA | 12227 | 0.2547 |
| hsa-miR-15b-5p | 57 | LM379035 | UAGCAGCACAUCAUGGUUUACA | 0.7328 | 0.2930 |
| hsa-miR-16-2-3p | 58 | LM380197 | CCAAUAUUACUGUGCUGCUUUA | 0.7448 | 0.6229 |
| hsa-miR-16-5p | 59 | LM378756 | UAGCAGCACGUAAAUAUUGGCG | 12208 | 0.5346 |
| hsa-miR-17-5p | 60 | LM378757 | CAAAGUGCUUACAGUGCAGGUAG | 10298 | 0.9090 |
| hsa-miR-181a-5p | 61 | LM378897 | AACAUUCAACGCUGUCGGUGAGU | 0.7900 | 0.4799 |
| hsa-miR-185-5p | 62 | LM379073 | UGGAGAGAAAGGCAGUUCCUGA | 11715 | 0.3799 |
| hsa-miR-186-5p | 63 | LM379074 | CAAAGAAUUCUCCUUUUGGGCU | 0.7588 | 0.3853 |
| hsa-miR-18a-5p | 64 | LM378759 | UAAGGUGCAUCUAGUGCAGAUAG | 0.7410 | 0.1600 |
| hsa-miR-18b-5p | 65 | LM379496 | UAAGGUGCAUCUAGUGCAGUUAG | 0.8291 | 0.4506 |
| hsa-miR-191-5p | 66 | LM379058 | CAACGGAAUCCCAAAAGCAGCUG | 0.8107 | 0.9090 |
| hsa-miR-192-5p | 67 | LM378867 | CUGACCUAUGAAUUGACAGCC | 0.9541 | 0.9758 |
| hsa-miR-193a-5p | 68 | LM380289 | UGGGUCUUUGCGGGCGAGAUGA | 0.7372 | 0.5625 |
| hsa-miR-194-5p | 69 | LM379078 | UGUAACAGCAACUCCAUGUGGA | 13271 | 0.1883 |
| hsa-miR-195-5p | 70 | LM379079 | UAGCAGCACAGAAAUAUUGGC | 10925 | 0.9312 |
| hsa-miR-197-3p | 71 | LM378872 | UUCACCACCUUCUCCACCCAGC | 12148 | 0.4688 |
| hsa-miR-199a-3p | 72 | LM378876 | ACAGUAGUCUGCACAUUGGUUA | 0.7168 | 0.5052 |
| hsa-miR-199a-5p | 73 | LM378875 | CCCAGUGUUCAGACUACCUGUUC | 0.6135 | 0.6147 |
| hsa-miR-19a-3p | 74 | LM378760 | UGUGCAAAUCUAUGCAAAACUGA | 0.7874 | 0.5052 |
| hsa-miR-19b-3p | 75 | LM378761 | UGUGCAAAUCCAUGCAAAACUGA | 12538 | 0.3347 |
| hsa-miR-200a-3p | 76 | LM379202 | UAACACUGUCUGGUAACGAUGU | 0.5255 | 0.0885 |
| hsa-miR-200c-3p | 77 | LM379140 | VAAUACUGCCGGGUAAUGAUGGA | 0.6815 | 0.3853 |
| hsa-miR-205-5p | 78 | LM378906 | UCCUUCAUUCCACCGGAGUCUG | 10074 | 0.8583 |
| hsa-miR-208a-3p | 79 | | AUAAGACGAGCAAAAAGCUUGU | undetectable | / |
| hsa-miR-20a-5p | 80 | LM378762 | UAAAGUGCUUAUAGUGCAGGUAG | 12370 | 0.3347 |
| hsa-miR-20b-5p | 81 | LM379497 | CAAAGUGCUCAUAGUGCAGGUAG | 0.7636 | 0.4506 |
| hsa-miR-210-3p | 82 | LM378907 | CUGUGCGUGUGACAGCGGCUGA | 0.7193 | 0.8629 |
| hsa-miR-2110 | 83 | LM381216. | UUGGGGAAACGGCCGCUGAGUG | 13304 | 0.1891 |
| hsa-miR-215-5p | 84 | LM378912. | AUGACCUAUGAAUUGACAGAC | 0.8163 | 0.6517 |
| hsa-miR-21-5p | 85 | LM378763 | UAGCUUAUCAGACUGAUGUUGA | 0.8964 | 0.5919 |
| hsa-miR-221-3p | 86 | LM378917 | AGCUACAUUGUCUGCUGGGUUUC | 0.0834 | 0.0372 |
| hsa-miR-222-3p | 87 | LM378918 | AGCUACAUCUGGCUACUGGGU | 0.8579 | 0.5052 |
| hsa-miR-223-3p | 88 | LM378919 | UGUCAGUUUGUCAAAUACCCCA | 0.7621 | 0.9758 |
| hsa-miR-223-5p | 89 | LM380248 | CGUGUAUUUGACAAGCUGAGUU | 11299 | 0.3996 |
| hsa-miR-22-3p | 90 | LM378764 | AAGCUGCCAGUUGAAGAACUGU | 0.5974 | 0.4834 |
| hsa-miR-22-5p | 91 | LM380174 | AGUUCUUCAGUGGCAAGCUUUA | 0.6710 | 0.5923 |
| hsa-miR-23a-3p | 92 | LM378765 | AUCACAUUGCCAGGGAUUUCC | 0.7596 | 0.5919 |
| hsa-miR-23b-3p | 93 | LM379036 | AUCACAUUGCCAGGGAUUACC | 0.6829 | 0.1600 |
| hsa-miR-24-3p | 94 | LM378767 | UGGCUCAGUUCAGCAGGAACAG | 0.8625 | 0.5919 |
| hsa-miR-25-3p | 95 | LM378768 | CAUUGCACUUGUCUCGGUCUGA | 16597 | 0.0003 |
| hsa-miR-26a-5p | 96 | LM378769 | UUCAAGUAAUCCAGGAUAGGCU | 0.7557 | 0.5346 |
| hsa-miR-26b-5p | 97 | LM378770 | UUCAAGUAAUUCAGGAUAGGU | 10200 | 0.8427 |
| hsa-miR-27a-3p | 98 | LM378771 | UUCACAGUGGCUAAGUUCCGC | 10476 | 0.8217 |
| hsa-miR-27b-3p | 99 | LM379037. | UUCACAGUGGCUAAGUUCUGC | 11180 | 0.3347 |
| hsa-miR-28-3p | 100 | LM380181 | CACUAGAUUGUGAGCUCCUGGA | 0.8876 | 0.7446 |
| hsa-miR-28-5p | 101 | LM378772 | AAGGAGCUCACAGUCUAUUGAG | 0.3251 | 0.1557 |
| hsa-miR-29a-3p | 102 | LM378773 | UAGCACCAUCUGAAAUCGGUUA | 10075 | 0.4799 |
| hsa-miR-29b-3p | 103 | LM378787 | UAGCACCAUUUGAAAUCAGUGUU | 0.9573 | 0.9336 |
| hsa-miR-29c-3p | 104 | LM379201 | UAGCACCAUUUGAAAUCGGUUA | 0.8377 | 0.7137 |
| hsa-miR-301a-3p | 105 | LM379208 | CAGUGCAAUAGUAUUGUCAAAGC | 10252 | 0.8845 |
| hsa-miR-30a-5p | 106 | LM378774 | UGUAAACAUCCUCGACUGGAAG | 0.8816 | 0.4799 |
| hsa-miR-30b-5p | 107 | LM379038 | UGUAAACAUCCUACACUCAGCU | 10422 | 0.9758 |
| hsa-miR-30c-5p | 108 | LM378886 | UGUAAACAUCCUACACUCUCAGC | 10951 | 0.5346 |
| hsa-miR-30d-5p | 109 | LM378887 | UGUAAACAUCCCCGACUGGAAG | 0.8342 | 0.7775 |
| hsa-miR-30e-3p | 110 | LM379213 | CUUUCAGUCGGAUGUUUACAGC | 0.8902 | 0.7975 |
| hsa-miR-30e-5p | 111 | LM379212 | UGUAAACAUCCUUGACUGGAAG | 0.8019 | 0.4799 |
| hsa-miR-320a | 112 | LM379081 | AAAAGCUGGGUUGAGAGGGCGA | 12150 | 0.9758 |
| hsa-miR-320b | 113 | LM380639 | AAAAGCUGGGUUGAGAGGGCAA | 14352 | 0.2930 |
| hsa-miR-320c | 114 | LM380640 | AAAAGCUGGGUUGAGAGGGU | 0.8162 | 0.2198 |
| hsa-miR-320d | 115 | LM380895 | AAAAGCUGGGUUGAGAGGA | 0.9512 | 0.7137 |
| hsa-miR-324-3p | 116 | LM379267 | ACUGCCCCAGGUGCUGCUGG | 0.6842 | 0.5052 |
| hsa-miR-324-5p | 117 | LM379266 | CGCAUCCCCUAGGGCAUUGGUGU | 0.8046 | 0.4506 |
| hsa-miR-32-5p | 118 | LM378777 | UAUUGCACAUUACUAAGUUGCA | 0.5000 | 0.2914 |
| hsa-miR-326 | 119 | LM379261 | CCUCUGGGCCCUUCCUCCAG | 10189 | 0.5784 |
| hsa-miR-328-3p | 120 | LM379257 | CUGGCCCUCUCUGCCCUUCCGU | 0.5122 | 0.3347 |
| hsa-miR-331-3p | 121 | LM379265 | GCCCCUGGGCCUAUCCUAGAA | 10209 | >0.9999 |
| hsa-miR-335-3p | 122 | LM380363 | UUUUUCAUUAUUGCUCCUGACC | 0.8393 | 0.6256 |
| hsa-miR-335-5p | 123 | LM379270 | UCAAGAGCAAUAACGAAAAAUGU | 0.6735 | 0.3962 |
| hsa-miR-338-3p | 124 | LM379268 | UCCAGCAUCAGUGAUUUUGUUG | 10632 | 0.9274 |
| hsa-miR-339-3p | 125 | LM380362 | UGAGCGCCUCGACGACAGAGCCG | 11124 | 0.7504 |
| hsa-miR-339-5p | 126 | LM379269 | UCCCUGUCCUCCAGGAGCUCACG | 0.6035 | 0.5408 |
| hsa-miR-33a-5p | 127 | LM378778 | GUGCAUUGUAGUUGCAUUGCA | 0.0480 | 0.1923 |
| hsa-miR-342-3p | 128 | LM379258 | UCUCACACAGAAAUCGCACCCGU | 0.8594 | 0.7137 |
| hsa-miR-34a-5p | 129 | LM378896 | UGGCAGUGUCUUAGCUGGUUGU | 0.7191 | 0.4680 |
| hsa-miR-361-5p | 130 | LM379218 | UUAUCAGAAUCUCCAGGGGUAC | 0.8130 | 0.9090 |
| hsa-miR-362-3p | 131 | LM380347 | AACACACCUAUUCAAGGAUUCA | 0.8524 | 0.7276 |
| hsa-miR-363-3p | 132 | LM379222 | AAUUGCACGGUAUCCAUCUGUA | 0.7229 | 0.4283 |
| hsa-miR-365a-3p | 133 | LM379224 | UAAUGCCCCUAAAAAUCCUUAU | 0.7774 | 0.4839 |
| hsa-miR-374a-5p | 134 | LM379233 | UUAUAAUACAACCUGAUAAGUG | 0.8463 | 0.4283 |
| hsa-miR-374b-5p | 135 | LM380517 | AUAUAAUACAACCUGCUAAGUG | 0.5887 | 0.0379 |
| hsa-miR-375 | 136 | LM379234 | UUUGUUCGUUCGGCUCGCGUGA | 0.8866 | 0.2198 |
| hsa-miR-376a-3p | 137 | LM379235 | AUCAUAGAGGAAAAUCCACGU | 0.5346 | 0.0623 |
| hsa-miR-376c-3p | 138 | LM379229 | AACAUAGAGGAAAUUCCACGU | 0.8026 | 0.6038 |
| hsa-miR-378a-3p | 139 | LM379238 | ACUGGACUUGGAGUCAGAAGG | 14878 | 0.2198 |
| hsa-miR-382-5p | 140 | LM379242 | GAAGUUGUUCGUGGUGGAUUCG | 0.7891 | 0.6038 |
| hsa-miR-409-3p | 141 | LM379542 | GAAUGUUGCUCGGUGAACCCCU | 0.7997 | 0.4731 |
| hsa-miR-421 | 142 | LM379897 | AUCAACAGACAUUAAUUGGGCGC | 0.2926 | 0.8689 |
| hsa-miR-423-3p | 143 | LM379468 | AGCUCGGUCUGAGGCCCCUCAGU | 0.7019 | 0.7775 |
| hsa-miR-423-5p | 144 | LM380368 | UGAGGGGCAGAGAGCGAGACUUU | 0.8289 | 0.3799 |
| hsa-miR-424-5p | 145 | LM379469 | CAGCAGCAAUUCAUGUUUUGAA | 0.6375 | 0.6229 |
| hsa-miR-425-3p | 146 | LM379471 | AUCGGGAAUGUCGUGUCCGCCC | 0.9544 | 0.8167 |
| hsa-miR-425-5p | 147 | LM379922 | AAUGACACGAUCACUCCCGUUGA | 0.5154 | 0.0140 |
| hsa-miR-451a | 148 | LM379535 | AAACCGUUACCAUUACUGAGUU | 0.9542 | 0.9090 |
| hsa-miR-454-3p | 149 | LM380029 | UAGUGCAAUAUUGCUUAUAGGGU | 0.8687 | 0.7137 |
| hsa-miR-483-5p | 150 | LM380378 | AAGACGGGAGGAAAGAAGGGAG | 0.8579 | 0.6575 |
| hsa-miR-484 | 151 | LM379708 | UCAGGCUCAGUCCCCUCCCGAU | 12267 | 0.2198 |
| hsa-miR-485-3p | 152 | LM379710 | GUCAUACACGGCUCUCCUCUCU | 0.2104 | 0.0858 |
| hsa-miR-486-5p | 153 | LM379711 | UCCUGUACUGAGCUGCCCCGAG | 13724 | 0.3799 |
| hsa-miR-495-3p | 154 | LM379774 | AAACAAACAUGGUGCACUUCUU | 0.5088 | 0.2073 |
| hsa-miR-497-5p | 155 | LM379777 | CAGCAGCACACUGUGGUUUGU | 0.4334 | 0.0611 |
| hsa-miR-501-3p | 156 | LM380388 | AAUGCACCCGGGCAAGGAUUCU | 11549 | >0.9999 |
| hsa-miR-502-3p | 157 | LM380389 | AAUGCACCUGGGCAAGGAUUCA | 11761 | 0.9312 |
| hsa-miR-505-3p | 158 | LM379800 | CGUCAACACUUGCUGGUUUCCU | 10740 | 0.6656 |
| hsa-miR-532-3p | 159 | LM380394 | CCUCCCACACCCAAGGCUUGCA | 0.8163 | 0.9090 |
| hsa-miR-532-5p | 160 | LM379811 | CAUGCCUUGAGUGUAGGACCGU | 0.7844 | 0.6517 |
| hsa-miR-543 | 161 | LM380516 | AAACAUUCGCGGUGCACUUCUU | 0.7224 | 0.2433 |
| hsa-miR-574-3p | 162 | LM379859 | CACGCUCAUGCACACACCCACA | 0.9071 | 0.8689 |
| hsa-miR-584-5p | 163 | LM379864 | UUAUGGUUUGCCUGGGACUGAG | 0.4297 | 0.2747 |
| hsa-miR-590-5p | 164 | LM379870 | GAGCUUAUUCAUAAAAGUGCAG | 0.9527 | 0.9090 |
| hsa-miR-629-5p | 165 | LM380419 | UGGGUUUACGUUGGGAGAACU | 0.9866 | 0.8614 |
| hsa-miR-652-3p | 166 | LM379886 | AAUGGCGCCACUAGGGUUGUG | 0.8554 | 0.8427 |
| hsa-miR-660-5p | 167 | LM379896 | UACCCAUUGCAUAUCGGAGUUG | 0.8299 | 0.1883 |
| hsa-miR-7-1-3p | 168 | LM380232 | CAACAAAUCACAGUCUGCCAUA | 0.9948 | 0.9766 |
| hsa-miR-7-5p | 169 | LM378893 | UGGAAGACUAGUGAUUUUGUUGU | 0.7801 | 0.4506 |
| hsa-miR-766-3p | 170 | LM380032 | ACUCCAGCCCCACAGCCUCAGC | 0.5761 | 0.2738 |
| hsa-miR-874-3p | 171 | LM380484 | CUGCCCUGGCCCGAGGGACCGA | 0.0390 | 0.0769 |
| hsa-miR-877-5p | 172 | LM380511 | GUAGAGGAGAUGGCGCAGGG | 13376 | 0.5846 |
| hsa-miR-885-5p | 173 | LM380509 | UCCAUUACACUACCCUGCCUCU | 13741 | 0.8629 |
| hsa-miR-92a-3p | 174 | LM378779 | UAUUGCACUUGUCCCGGCCUGU | 16511 | 0.0623 |
| hsa-miR-92b-3p | 175 | LM379854 | UAUUGCACUCGUCCCGGCCUCC | 16259 | 0.3078 |
| hsa-miR-93-3p | 176 | LM380188 | ACUGCUGAGCUAGCACUUCCCG | 14462 | 0.6038 |
| hsa-miR-93-5p | 177 | LM378780 | CAAAGUGCUGUUCGUGCAGGUAG | 11092 | 0.4283 |
| hsa-miR-99a-5p | 178 | LM378784 | AACCCGUAGAUCCGAUCUUGUG | 0.8854 | 0.4791 |
| hsa-miR-99b-5p | 179 | LM379209 | CACCCGUAGAACCGACCUUGCG | 11308 | 0.8832 |

TABLE 6
AUC (Area Under Curve). Sensitivity and specificity
of 7 identified miRNAs in osteoporotic women.

| | | | | Sensi- | Speci- |
|---|---|---|---|---|---|
| | | | Youden's | tivity | ficity |
| miRNA | AUC [95% CI] | p-value | index | (%) | (%) |
| hsa-miR- | 0.802 | 0.031 | 0.556 | 77.78 | 77.78 |
| 145-5p | [0.596-1.009] | | | | |
| hsa-miR- | 1.000 | 0.001 | 0.875 | 87.50 | 100.00 |
| 25-3p | [1.000-1.000] | | | | |
| hsa-miR- | 0.857 | 0.017 | 0.667 | 100.00 | 66.67 |
| 126-5p | [0.660-1.054] | | | | |
| hsa-miR- | 0.893 | 0.011 | 0.750 | 100.00 | 75.00 |
| 146a-5p | [0.713-1.072] | | | | |
| hsa-miR- | 0.824 | 0.039 | 0.667 | 100.00 | 66.67 |
| 221-3p | [0.603-1.045] | | | | |
| hsa-miR- | 0.812 | 0.036 | 0.625 | 87.50 | 75.00 |
| 374b-5p | [0.592-1.033] | | | | |
| hsa-miR- | 0.875 | 0.015 | 0.607 | 85.71 | 75.00 |
| 425-5p | [0.697-1.053] | | | | |

### Example 3: MiRNA and ASMMI Association

To assess the relationship of identified miRNA level with skeletal muscle mass, association analyses with ASMMI, as index of skeletal muscle mass, were performed.

Five clinical variables were chosen as covariates: age, BMI, fat, fat percentage, T-score. In univariate analysis, two miRNAs, hsa-miR-126-5p SEQ ID NO: 25 (p-value=0.007) and hsa-miR-146a-5p SEQ ID NO: 45 (p-value=0.006), were significantly associated with ASMMI (Table 7). In bivariate analysis, considering miRNAs levels as continuous variables, hsa-miR-126-5p SEQ ID NO: 25 and hsa-miR-25-3p SEQ ID NO: 95 were associated with ASMMI when adjusted for age (p-value=0.042 and p-value=0.049, respectively), and hsa-miR-146a-5p when adjusted for age (p-value=0.026), fat percentage (p-value=0.022) and T-score (p-value=0.025) (Table 7). Multivariate analyses have shown that none of the identified miRNAs have been independently associated with ASMMI (Table 7).

TABLE 7
Association analysis of the identified miRNAs with ASMMI.
ASSOCIATION miRNA with ASMMI

| | | | Estimate | | Estimate | | Estimate | | Estimate | | Estimate | |
|---|---|---|---|---|---|---|---|---|---|---|---|---|
| | | | (SE) | | (SE) | | (SE) | | (SE) | | (SE) | |
| | Estimate | | AGE | | BMI | | FAT | | % FAT | | T. SCORE | |
| miRNA | (SE) | p-value | adjusted | p-value | adjusted | p-value | adjusted | p-value | adjusted | p-value | adjusted | p-value |
| hsa-miR-221-3p | 0.15 | 0.189 | 0.15 | 0.187 | 0.10 | 0.497 | 0.13 | 0.292 | 0.13 | 0.226 | 0.13 | 0.275 |
| | (0.11) | | (0.11) | | (0.15) | | (0.12) | | (0.10) | | (0.11) | |
| hsa-miR-374b-5p | 0.07 | 0.085 | 0.07 | 0.051 | 0.04 | 0.399 | 0.06 | 0.152 | 0.06 | 0.089 | 0.06 | 0.117 |
| | (0.04) | | (0.00) | | (0.05) | | (0.04) | | (0.04) | | (0.04) | |
| hsa-miR-145-5p | −0.06 | 0.671 | −0.06 | 0.645 | −0.11 | 0.532 | −0.13 | 0.353 | −0.06 | 0.652 | −0.04 | 0.763 |
| | (0.13) | | (0.13) | | (0.17) | | (0.13) | | (0.13) | | (0.13) | |
| hsa-miR-25-3p | −1.08 | 0.051 | −1.11 | 0.049 | −0.82 | 0.233 | −0.99 | 0.088 | −1.07 | 0.057 | −1.08 | 0.057 |
| | (0.53) | | (0.53) | | (0.67) | | (0.56) | | (0.53) | | (0.54) | |
| hsa-mi-146a-5p | 0.14 | 0.021 | 0.14 | 0.026 | 0.04 | 0.531 | 0.12 | 0.063 | 0.14 | 0.022 | 0.14 | 0.025 |
| | (0.06) | | (0.06) | | (0.07) | | (0.06) | | (0.06) | | (0.06) | |
| hsa-miR-126-5p | 0.15 | 0.049 | 0.14 | 0.042 | 0.08 | 0.379 | 0.12 | 0.132 | 0.15 | 0.056 | 0.13 | 0.077 |
| | (0.07) | | (0.07) | | (0.09) | | (0.08) | | (0.07) | | (0.07) | |
| hsa-miR-425-3p | 0.12 | 0.066 | 0.12 | 0.071 | 0.14 | 0.107 | 0.14 | 0.054 | 0.14 | 0.054 | 0.11 | 0.088 |
| | (0.06) | | (0.06) | | (0.08) | | (0.07) | | (0.07) | | (0.06) | |

### Example 4: Evaluation of miRNA-Panel as Biomarker for Skeletal Muscle Mass Decrease

Combining the 2 miRNAs associated with ASMMI (hsa-miR-126-5p SEQ ID NO: 25, hsa-miR-146a-5p SEQ ID NO: 45), the AUC was 0.864 (p=0.015), with a sensitivity and a specificity of 77.78 and 100, respectively (Table 8). Each identified miRNA was added to this miRNA-panel (hsa-miR-126-5p, hsa-miR-146a-5p) to evaluate the diagnostic potential of combined miRNAs. The obtained AUC range from 0.704 to 0.914, with sensitivity and specificity range from 55.56 to 88.89 and from 66.67 to 100, respectively, as shown in Table 5. The highest AUC was obtained combining hsa-miR-126-5p, hsa-miR-146a-5p and hsa-miR-425-5p (AUC=0.914; sensitivity=77.78; specificity=100) or combining hsa-miR-126-5p, hsa-miR-146a-5p, hsa-miR-145-5p and hsa-miR-25a-5p (AUC=0.901; sensitivity=88.89; specificity=100) (Table 8).

TABLE 8
ROC curve analysis of combination of the seven identified miRNAs. The combinations
were built considering hsa-miR-126-5p and hsa-miR-146a-5p panel fixed.

| | AUC | | Youden's | Sensitivity | Specificity |
|---|---|---|---|---|---|
| miRNAs | [95% CI] | P value | index | (%) | (%) |
| miR-146a-5p-miR-126-5p | 0.864 | 0.009 | 0.778 | 77.78 | 100 |
| | [0.679-1.049] | | | | |
| miR-146a-5p-miR-126-5p, | 0.840 | 0.015 | 0.778 | 77.78 | 100 |
| miR-221-3p | [0.627-1.052] | | | | |
| miR-146a-5p-miR-126-5p, | 0.827 | 0.0193 | 0.667 | 77.78 | 88.89 |
| miR-374b-5p | [0.619-1.036] | | | | |
| miR-146a-5p-miR-126-5p, | 0.704 | 0.145 | 0.3337 | 66.67 | 66.67 |
| miR-145-5p | [0.459-0.948] | | | | |
| miR-146a-5p-miR-126-5p, | 0.839 | 0.015 | 0.778 | 77.78 | 100 |
| miR-25-3p | [0.619-1.059] | | | | |
| miR-146a-5p-miR-126-5p, | 0.914 | 0.003 | 0.778 | 77.78 | 100 |
| miR-425-5p | [0.779-1.047] | | | | |
| miR-146a-5p-miR-126-5p, | 0.815 | 0.024 | 0.556 | 77.78 | 77.78 |
| miR-221-3p, miR-374b-5p | [0.603-1.026] | | | | |
| miR-146a-5p-miR-126-5p, | 0.740 | 0.085 | 0.556 | 66.67 | 88.89 |
| miR-221-3p, miR-145-5p | [0.497-0.984] | | | | |
| miR-146a-5p-miR-126-5p, | 0.839 | 0.015 | 0.778 | 77.78 | 100 |
| miR-221-3p, miR-25-3p | [0.619-1.059] | | | | |
| miR-146a-5p-miR-126-5p, | 0.864 | 0.009 | 0.778 | 77.78 | 100 |
| miR-221-3p, miR-425-5p | [0.676-1.052] | | | | |
| miR-146a-5p-miR-126-5p, | 0.790 | 0.038 | 0.556 | 66.67 | 88.89 |
| miR-374b-5p, miR-145-5p | [0.577-1.003] | | | | |
| miR-146a-5p-miR-126-5p, | 0.802 | 0.030 | 0.667 | 77.78 | 88.89 |
| miR-374b-5p, miR-25-3p | [0.575-1.030] | | | | |
| miR-146a-5p-miR-126-5p, | 0.876 | 0.007 | 0.667 | 77.78 | 88.89 |
| miR-374b-5p, miR-425-5p | [0.716-1.037] | | | | |
| miR-146a-5p-miR-126-5p, | 0.901 | 0.004 | 0.889 | 88.89 | 100 |
| miR-145-5p, miR-25-3p | [0.716-1.086] | | | | |
| miR-146a-5p-miR-126-5p, | 0.839 | 0.015 | 0.556 | 88.89 | 66.67 |
| miR-145-5p, miR-425-5p | [0.656-1.023] | | | | |
| miR-146a-5p-miR-126-5p, | 0.839 | 0.015 | 0.778 | 77.78 | 100 |
| miR-25-3p, miR-425-5p | [0.620-1.059] | | | | |
| miR-146a-5p-miR-126-5p, | 0.790 | 0.038 | 0.556 | 66.67 | 88.89 |
| miR-221-3p, miR-374b-5p, | [0.568-1.012] | | | | |
| miR-145-5p | | | | | |
| miR-146a-5p-miR-126-5p, | 0.827 | 0.019 | 0.778 | 77.78 | 100 |
| miR-221-3p, miR-374b-5p, | [0.599-1.055] | | | | |
| miR-25-3p | | | | | |
| miR-146a-5p-miR-126-5p, | 0.852 | 0.011 | 0.667 | 88.89 | 77.78 |
| miR-221-3p, miR-374b-5p, | [0.664-1.039] | | | | |
| miR-425-5p | | | | | |
| miR-146a-5p-miR-126-5p, | 0.852 | 0.011 | 0.667 | 88.89 | 77.78 |
| miR-374b-5p, miR-145-5p, | [0.641-1.063] | | | | |
| miR-25-3p | | | | | |
| miR-146a-5p-miR-126-5p, | 0.852 | 0.011 | 0.556 | 88.89 | 66.67 |
| miR-374b-5p, miR-145-5p, | [0.676-1.028] | | | | |
| miR-25-3p | | | | | |
| miR-146a-5p-miR-126-5p, | 0.876 | 0.007 | 0.778 | 77.78 | 100 |
| miR-145-5p, miR-25-3p, | [0.670-1.083] | | | | |
| miR-425-5p | | | | | |
| miR-146a-5p-miR-126-5p, | 0.889 | 0.005 | 0.778 | 77.78 | 100 |
| miR-145-5p, miR-25-3p, | [0.702-1.076] | | | | |
| miR-221-3p | | | | | |
| mIR-146a-5p-miR-126-5p, | 0.840 | 0.015 | 0.778 | 77.78 | 100 |
| miR-25-3p, miR-425-5p, | [0.620-1.059] | | | | |
| miR-221-3p | | | | | |
| miR-146a-5p-miR-126-5p, | 0.840 | 0.015 | 0.778 | 77.78 | 100 |
| miR-25-3p, miR-425-5p, | [0.620-1.059] | | | | |
| miR-374b-5p | | | | | |
| miR-146a-5p-miR-126-5p, | 0.779 | 0.047 | 0.556 | 55.56 | 100 |
| miR-425-5p, miR-221-3p, | [0.557-0.999] | | | | |
| miR-145-5p | | | | | |
| miR-146a-5p-miR-126-5p, | 0.840 | 0.015 | 0.778 | 77.78 | 100 |
| miR-221-3p, miR-374b-5p, | [0.620-1.059] | | | | |
| miR-145-5p, miR-25-3p | | | | | |
| miR-146a-5p-miR-126-5p, | 0.828 | 0.019 | 0.556 | 66.67 | 88.89 |
| miR-221-3p, miR-374b-5p, | [0.629-1.025] | | | | |
| miR-145-5p, miR-425-5p | | | | | |
| miR-146a-5p-miR-126-5p, | 0.828 | 0.019 | 0.778 | 77.78 | 100 |
| miR-221-3p, miR-374b-5p, | [0.599-1.055] | | | | |
| miR-25-3p, miR-425-5p | | | | | |
| miR-146a-5p-miR-126-5p, | 0.865 | 0.009 | 0.778 | 77.78 | 100 |
| miR-221-3p, miR-145-5p, | [0.655-1.073] | | | | |
| miR-25-3p, miR-425-5p | | | | | |
| miR-146a-5p-miR-126-5p, | 0.840 | 0.015 | 0.778 | 77.78 | 100 |
| miR-374b-5p, miR-145-5p, | [0.620-1.059] | | | | |
| miR-25-3p, miR-425-5p | | | | | |

Combining the same 2 miRNAs associated with ASMMI (hsa-miR-126-5p SEQ ID NO: 25, hsa-miR-146a-5p SEQ ID NO: 45), the statistical analysis has been repeated adding the T-score, an index of bone mineral density, as a predictor. Although the women included in the study are all osteoporotic with a T-scores −2.5 SD, the adjustment of the logistic model made it possible to eliminate the effect/influence of bone density. The AUC was 0.926 (p=0.0023), with a sensitivity and a specificity of 88.9 (Table 9). Each identified miRNA was added to this miRNA-panel (hsa-miR-126-5p, hsa-miR-146a-5p). Each combination shows an increase of the AUC, performing the T-score adjusted statistical analysis, as shown in Table 5. The highest AUC was obtained combining hsa-miR-126-5p, hsa-miR-146a-5p, hsa-miR-425-5p, has-miR-145-5p, has-miR-221-3p, hsa-miR-25-3p (AUC=0.988; p=0.0005) (Table 9).

TABLE 9
ROC curve analysis of combination of the seven identified miRNAs, adding the T-score as predictor.
The combinations were built considering hsa-miR-126-5p and hsa-miR-146a-5p panel fixed.

| miRNA | AUC | pvalue | Yindex | sensitivity | specificity |
|---|---|---|---|---|---|
| mirR-146a-5p-miR-126-5p | 0.926 | 0.0023 | 0.778 | 88.9 | 88.9 |
| | (0.805 to 1.00) | | | | |
| mirR-146a-5p-miR-126-5p, miR-221-3p | 0.926 | 0.0023 | 0.778 | 88.9 | 88.9 |
| | (0.805 to 1.00) | | | | |
| mirR-146a-5p-miR-126-5p, miR-374b-5p | 0.926 | 0.0023 | 0.778 | 88.9 | 88.9 |
| | (0.805 to 1.00) | | | | |
| mirR-146a-5p-miR-126-5p, miR-145-5p | 0.938 | 0.0017 | 0.778 | 88.9 | 88.9 |
| | (0.832-1.00) | | | | |
| mirR-146a-5p-miR-126-5p, miR-25-3p | 0.951 | 0.0013 | 0.778 | 77.8 | 100 |
| | (0.857 to 1.00) | | | 88.9 | 88.9 |
| mirR-146a-5p-miR-126-5p, miR-425-5p | 0.938 | 0.0017 | 0.778 | 77.8 | 100 |
| | (0.827-1.00) | | | 88.9 | 88.9 |
| mirR-146a-5p-miR-126-5p, miR-221-3p, | 0.926 | 0.0023 | 0.778 | 88.9 | 88.9 |
| miR-374b-5p | (0.805 to 1.00) | | | | |
| mirR-146a-5p-miR-126-5p, miR-221-3p, | 0.951 | 0.0013 | 0.778 | 77.8 | 100 |
| miR-145-5p | (0.857 to 1.00) | | | 88.9 | 88.9 |
| mirR-146a-5p-miR-126-5p, miR-221-3p, | 0.963 | 0.0009 | 0.778 | 77.8 | 100 |
| miR-25-3p | (0.886 to 1.00) | | | 88.9 | 88.9 |
| | | | | 100 | 77.8 |
| mirR-146a-5p-miR-126-5p, miR-221-3p, | 0.938 | 0.0017 | 0.778 | 77.8 | 100 |
| miR-425-5p | (0.827-1.00) | | | 88.9 | 88.9 |
| mirR-146a-5p-miR-126-5p, miR-374b-5p, | 0.951 | 0.0013 | 0.778 | 77.8 | 100 |
| miR-145-5p | (0.857 to 1.00) | | | 88.9 | 88.9 |
| mirR-146a-5p-miR-126-5p, miR-374b-5p, | 0.975 | 0.0007 | 0.889 | 100 | 88.9 |
| miR-25-3p | (0.914 to 1.00) | | | | |
| mirR-146a-5p-miR-126-5p, miR-374b-5p, | 0.938 | 0.0017 | 0.778 | 77.8 | 100 |
| miR-425-5p | (0.827-1.00) | | | 88.9 | 88.9 |
| mirR-146a-5p-miR-126-5p, miR-145-5p, | 0.951 | 0.0013 | 0.778 | 77.8 | 100 |
| miR-25-3p | (0.857 to 1.00) | | | 88.9 | 88.9 |
| mirR-146a-5p-miR-126-5p, miR-145-5p, | 0.938 | 0.0017 | 0.778 | 77.8 | 100 |
| miR-425-5p | (0.827-1.00) | | | 88.9 | 88.9 |
| mirR-146a-5p-miR-126-5p, miR-25-3p, | 0.951 | 0.0013 | 0.778 | 77.8 | 100 |
| miR-425-5p | (0.857 to 1.00) | | | 88.9 | 88.9 |
| mirR-146a-5p-miR-126-5p, miR-221-3p, | 0.951 | 0.0013 | 0.778 | 77.8 | 100 |
| miR-374b-5p, miR-145-5p | (0.857 to 1.00) | | | 88.9 | 88.9 |
| mirR-146a-5p-miR-126-5p, miR-221-3p, | 0.975 | 0.0007 | 0.889 | 100 | 88.9 |
| miR-374b-5p, miR-25-3p | (0.914 to 1.00) | | | | |
| mirR-146a-5p-miR-126-5p, miR-221-3p, | 0.938 | 0.0017 | 0.778 | 77.8 | 100 |
| miR-374b-5p, miR-425-5p | (0.827-1.00) | | | 88.9 | 88.9 |
| mirR-146a-5p-miR-126-5p, miR-374b-5p, | 0.963 | 0.0009 | 0.778 | 77.8 | 100 |
| miR-145-5p, miR-25-3p | (0.886 to 1.00) | | | 88.9 | 88.9 |
| | | | | 100 | 77.8 |
| mirR-146a-5p-miR-126-5p, miR-374b-5p, | 0.938 | 0.0017 | 0.778 | 77.8 | 100 |
| miR-145-5p, miR-425-5p | (0.827-1.00) | | | 88.9 | 88.9 |
| | | 0.0013 | 0.778 | 77.8 | 100 |
| miR-146a-5p-miR-126-5p, miR-145-5p, | 0.951 | | | 88.9 | 88.9 |
| miR-25-3p, miR-425-5p | (0.857 to 1.00) | | | | |
| miR-146a-5p-miR-126-5p, miR-145-5p, | 0.975 | 0.0007 | 0.889 | 88.9 | 100 |
| miR-25-3p, miR-221-3p | (0.914 to 1.00) | | | | |
| miR-146a-5p-miR-126-5p, miR-145-5p, | 0.938 | 0.0017 | 0.778 | 77.8 | 100 |
| miR-425-3p, miR-221-3p | (0.827-1.00) | | | 88.9 | 88.9 |
| miR-146a-5p-miR-126-5p, miR-25-3p, | 0.963 | 0.0009 | 0.778 | 77.8 | 100 |
| miR-425-5p, miR-221-3p | (0.886 to 1.00) | | | 88.9 | 88.9 |
| | | | | 100 | 77.8 |
| miR-146a-5p-miR-126-5p, miR-25-3p, | 0.975 | 0.0007 | 0.889 | 100 | 88.9 |
| miR-425-5p, miR-374b-5p | (0.914 to 1.00) | | | | |
| miR-146a-5p-miR-126-5p, miR-221-3p, | 0.975 | 0.0007 | 0.889 | 100 | 88.9 |
| miR-374b-5p, miR-145-5p, miR-25-3p | (0.914 to 1.00) | | | | |
| miR-146a-5p-miR-126-5p, miR-221-3p, | 0.938 | 0.0017 | 0.778 | 77.8 | 100 |
| miR-374b-5p, miR-145-5p, miR-425-5p | (0.827-1.00) | | | 88.9 | 88.9 |
| miR-146a-5p-miR-126-5p, miR-221-3p, | 0.975 | 0.0007 | 0.889 | 100 | 88.9 |
| miR-374b-5p, miR-25-3p, miR-425-5p | (0.914 to 1.00) | | | | |
| miR-146a-5p-miR-126-5p, miR-374b-5p, | 0.975 | 0.0007 | 0.889 | 100 | 88.9 |
| miR-145-5p, miR-25-3p, miR-425-5p | (0.914 to 1.00) | | | | |
| miR-146a-5p-miR-126-5p, miR-221-3p, | 0.988 | 0.0005 | 0.889 | 88.9 | 100 |
| miR-145-5p, miR-25-3p, miR-425-5p | 0.949 to 1.00) | | | 100 | 88.9 |
| miR-146a-5p-miR-126-5p, miR-221-3p, | 0.975 | 0.0007 | 0.889 | 100 | 88.9 |
| miR-374b-5p, miR-145-5p, miR-25-3p, | (0.914 to 1.00) | | | | |
| miR-425-5p | | | | | |

### REFERENCES

1. Nih Consensus Development Panel on Osteoporosis Prevention, D.; Therapy. Osteoporosis prevention, diagnosis, and therapy. Jama 2001, 285, 785-795, doi: 10.1001/jama.285.6.785.
2. Fielding, R. A.; Vellas, B.; Evans, W. J.; Bhasin, S.; Morley, J. E.; Newman, A. B.; Abellan van Kan, G.; Andrieu, S.; Bauer, J.; Breuille, D.; et al. Sarcopenia: an undiagnosed condition in older adults. Current consensus definition: prevalence, etiology, and consequences. International working group on sarcopenia. Journal of the American Medical Directors Association 2011, 12, 249-256, doi: 10.1016/j.jamda.2011.01.003.
3. Hirschfeld, H. P.; Kinsella, R.; Duque, G. Osteosarcopenia: where bone, muscle, and fat collide. Osteoporosis international: a journal established as result of cooperation between the European Foundation for Osteoporosis and the National Osteoporosis Foundation of the USA 2017, 28, 2781-2790, doi: 10.1007/s00198-017-4151-8.
4. Hernlund, E.; Svedbom, A.; Ivergard, M.; Compston, J.; Cooper, C.; Stenmark, J.; McCloskey, E. V.; Jonsson, B.; Kanis, J. A. Osteoporosis in the European Union: medical management, epidemiology and economic burden. A report prepared in collaboration with the International Osteoporosis Foundation (IOF) and the European Federation of Pharmaceutical Industry Associations (EFPIA). Archives of osteoporosis 2013, 8, 136, doi: 10.1007/s11657-013-0136-1.
5. Cruz-Jentoft, A. J.; Baeyens, J. P.; Bauer, J. M.; Boirie, Y.; Cederholm, T.; Landi, F.; Martin, F. C.; Michel, J. P.; Rolland, Y.; Schneider, S. M.; et al. Sarcopenia: European consensus on definition and diagnosis: Report of the European Working Group on Sarcopenia in Older People. Age and ageing 2010, 39, 412-423, doi: 10.1093/ageing/afq034.
6. Dam, T. T.; Peters, K. W.; Fragala, M.; Cawthon, P. M.; Harris, T. B.; McLean, R.; Shardell, M.; Alley, D. E.; Kenny, A.; Ferrucci, L.; et al. An evidence-based comparison of operational criteria for the presence of sarcopenia. The journals of gerontology. Series A, Biological sciences and medical sciences 2014, 69, 584-590, doi: 10.1093/gerona/glu013.
7. Beaudart, C.; Rizzoli, R.; Bruyere, O.; Reginster, J. Y.; Biver, E. Sarcopenia: burden and challenges for public health. Archives of public health=Archives belges de sante publique 2014, 72, 45, doi: 10.1186/2049-3258-72-45.
8. Assessment of fracture risk and its application to screening for postmenopausal osteoporosis. Report of a WHO Study Group. World Health Organization technical report series 1994, 843, 1-129.
9. Fuggle, N.; Shaw, S.; Dennison, E.; Cooper, C. Sarcopenia. Best practice & research. Clinical rheumatology 2017, 31, 218-242, doi: 10.1016/j.berh.2017.11.007.
10. Kanis, J. A.; Harvey, N. C.; Johansson, H.; Oden, A.; Leslie, W. D.; McCloskey, E. V. FRAX and fracture prediction without bone mineral density. Climacteric: the journal of the International Menopause Society 2015, 18 Suppl 2, 2-9, doi: 10.3109/13697137.2015.1092342.
11. Malmstrom, T. K.; Miller, D. K.; Simonsick, E. M.; Ferrucci, L.; Morley, J. E. SARC-F: a symptom score to predict persons with sarcopenia at risk for poor functional outcomes. Journal of cachexia, sarcopenia and muscle 2016, 7, 28-36, doi: 10.1002/jcsm.12048.
12. Fathi, M.; Heshmat, R.; Ebrahimi, M.; Salimzadeh, A.; Ostovar, A.; Fathi, A.; Razi, F.; Nabipour, I.; Moghaddassi, M.; Shafiee, G. Association between biomarkers of bone health and osteosarcopenia among Iranian older people: The Bushehr Elderly Health (BEH) program. BMC geriatrics 2021, 21, 654, doi: 10.1186/s12877-021-02608-w.
13. Poggiogalle, E.; Cherry, K. E.; Su, L. J.; Kim, S.; Myers, L.; Welsh, D. A.; Jazwinski, S. M.; Ravussin, E. Body Composition, IGF1 Status, and Physical Functionality in Nonagenarians: Implications for Osteosarcopenia. Journal of the American Medical Directors Association 2019, 20, 70-75 e72, doi: 10.1016/j.jamda.2018.07.007.
14. Gao, Y.; Patil, S.; Qian, A. The Role of MicroRNAs in Bone Metabolism and Disease. International journal of molecular sciences 2020, 21, doi: 10.3390/ijms21176081.
15. Hensley, A. P.; McAlinden, A. The role of microRNAs in bone development. Bone 2021, 143, 115760, doi: 10.1016/j.bone.2020.115760.
16. Sjogren, R. J. O.; Lindgren Niss, M. H. L.; Krook, A. Skeletal Muscle microRNAs: Roles in Differentiation, Disease and Exercise. In Hormones, Metabolism and the Benefits of Exercise, Spiegelman, B., Ed.; Cham (CH), 2017; pp. 67-81.
17. Condrat, C. E.; Thompson, D. C.; Barbu, M. G.; Bugnar, O. L.; Boboc, A.; Cretoiu, D.; Suciu, N.; Cretoiu, S. M.; Voinea, S. C. miRNAs as Biomarkers in Disease: Latest Findings Regarding Their Role in Diagnosis and Prognosis. Cells-Basel 2020, 9, doi: 10.3390/cells9020276.
18. Yin, J.; Qian, Z.; Chen, Y.; Li, Y.; Zhou, X. MicroRNA regulatory networks in the pathogenesis of sarcopenia. Journal of cellular and molecular medicine 2020, 24, 4900-4912, doi: 10.1111/jcmm.15197.
19. Brown, D. M.; Goljanek-Whysall, K. microRNAs: Modulators of the underlying pathophysiology of sarcopenia? Ageing research reviews 2015, 24, 263-273, doi: 10.1016/j.arr.2015.08.007.
20. Bottani, M.; Banfi, G.; Lombardi, G. Perspectives on miRNAs as Epigenetic Markers in Osteoporosis and Bone Fracture Risk: A Step Forward in Personalized Diagnosis. Frontiers in genetics 2019, 10, 1044, doi: 10.3389/fgene.2019.01044.
21. Materozzi, M.; Merlotti, D.; Gennari, L.; Bianciardi, S. The Potential Role of miRNAs as New Biomarkers for Osteoporosis. International journal of endocrinology 2018, 2018, 2342860, doi: 10.1155/2018/2342860.
22. Donati, S.; Ciuffi, S.; Palmini, G.; Brandi, M. L. Circulating miRNAs: A New Opportunity in Bone Fragility. Biomolecules 2020, 10, doi: 10.3390/biom10060927.
23. Chen, Z.; Bemben, M. G.; Bemben, D. A. Bone and muscle specific circulating microRNAs in postmenopausal women based on osteoporosis and sarcopenia status. Bone 2019, 120, 271-278, doi: 10.1016/j.bone.2018.11.001.
24. He, N.; Zhang, Y. L.; Zhang, Y.; Feng, B.; Zheng, Z.; Wang, D.; Zhang, S.; Guo, Q.; Ye, H. Circulating MicroRNAs in Plasma Decrease in Response to Sarcopenia in the Elderly. Frontiers in genetics 2020, 11, 167, doi: 10.3389/fgene.2020.00167.
25. Jung, H. J.; Lee, K. P.; Kwon, K. S.; Suh, Y. MicroRNAs in Skeletal Muscle Aging: Current Issues and Perspectives. The journals of gerontology. Series A, Biological sciences and medical sciences 2019, 74, 1008-1014, doi: 10.1093/gerona/gly207.

1. A method for the in vitro diagnosis, prognosis and/or treatment monitoring of osteosarcopenia, wherein the method comprises the steps:
a) making available a test sample from the subject;
b) collecting the microRNA (miRNA) contained in the test sample;
c) determining the expression profile of a predetermined set of miRNA;
d) comparing said expression profile to one or several reference expressions profiles,
wherein the comparison of said determined expression profile to said one or several reference expression profiles allows for the diagnosis, prognosis and/or treatment monitoring of the disease.

2. The method according to claim 1, wherein said expression profile is determined of miRNAs selected from the group consisting of hsa-let-7a-5p, hsa-let-7b-3p, hsa-let-7b-5p, hsa-let-7c-5p, hsa-let-7d-3p, hsa-let-7d-5p, hsa-let-7e-5p, hsa-let-7f-5p, hsa-let-7g-5p, hsa-let-7i-5p, hsa-miR-1-3p, hsa-miR-100-5p, hsa-miR-101-3p, hsa-miR-103a-3p, hsa-miR-106a-5p, hsa-miR-106b-3p, hsa-miR-106b-5p, hsa-miR-107, hsa-miR-10b-5p, hsa-miR-122-5p, hsa-miR-125a-5p, hsa-miR-125b-5p, hsa-miR-1260a, hsa-miR-126-3p, hsa-miR-126-5p, hsa-miR-127-3p, hsa-miR-128-3p, hsa-miR-130a-3p, hsa-miR-130b-3p, hsa-miR-132-3p, hsa-miR-133a-3p, hsa-miR-133b, hsa-miR-136-3p, hsa-miR-136-5p, hsa-miR-139-5p, hsa-miR-140-3p, hsa-miR-140-5p, hsa-miR-141-3p, hsa-miR-142-3p, hsa-miR-142-5p, hsa-miR-143-3p, hsa-miR-144-3p, hsa-miR-144-5p, hsa-miR-145-5p, hsa-miR-146a-5p, hsa-miR-146b-5p, hsa-miR-148a-3p, hsa-miR-148b-3p, hsa-miR-150-5p, hsa-miR-151a-3p, hsa-miR-151a-5p, hsa-miR-152-3p, hsa-miR-154-5p, hsa-miR-155-5p, hsa-miR-15a-5p, hsa-miR-15b-3p, hsa-miR-15b-5p, hsa-miR-16-2-3p, hsa-miR-16-5p, hsa-miR-17-5p, hsa-miR-181a-5p, hsa-miR-185-5p, hsa-miR-186-5p, hsa-miR-18a-5p, hsa-miR-18b-5p, hsa-miR-191-5p, hsa-miR-192-5p, hsa-miR-193a-5p, hsa-miR-194-5p, hsa-miR-195-5p, hsa-miR-197-3p, hsa-miR-199a-3p, hsa-miR-199a-5p, hsa-miR-19a-3p, hsa-miR-19b-3p, hsa-miR-200a-3p, hsa-miR-200c-3p, hsa-miR-205-5p, hsa-miR-208a-3p, hsa-miR-20a-5p, hsa-miR-20b-5p, hsa-miR-210-3p, hsa-miR-2110, hsa-miR-215-5p, hsa-miR-21-5p, hsa-miR-221-3p, hsa-miR-222-3p, hsa-miR-223-3p, hsa-miR-223-5p, hsa-miR-22-3p, hsa-miR-22-5p, hsa-miR-23a-3p, hsa-miR-23b-3p, hsa-miR-24-3p, hsa-miR-25-3p, hsa-miR-26a-5p, hsa-miR-26b-5p, hsa-miR-27a-3p, hsa-miR-27b-3p, hsa-miR-28-3p, hsa-miR-28-5p, hsa-miR-29a-3p, hsa-miR-29b-3p, hsa-miR-29c-3p, hsa-miR-301a-3p, hsa-miR-30a-5p, hsa-miR-30b-5p, hsa-miR-30c-5p, hsa-miR-30d-5p, hsa-miR-30e-3p, hsa-miR-30e-5p, hsa-miR-320a, hsa-miR-320b, hsa-miR-320c, hsa-miR-320d, hsa-miR-324-3p, hsa-miR-324-5p, hsa-miR-32-5p, hsa-miR-326, hsa-miR-328-3p, hsa-miR-331-3p, hsa-miR-335-3p, hsa-miR-335-5p, hsa-miR-338-3p, hsa-miR-339-3p, hsa-miR-339-5p, hsa-miR-33a-5p, hsa-miR-342-3p, hsa-miR-34a-5p, hsa-miR-361-5p, hsa-miR-362-3p, hsa-miR-363-3p, hsa-miR-365a-3p, hsa-miR-374a-5p, hsa-miR-374b-5p, hsa-miR-375, hsa-miR-376a-3p, hsa-miR-376c-3p, hsa-miR-378a-3p, hsa-miR-382-5p, hsa-miR-409-3p, hsa-miR-421, hsa-miR-423-3p, hsa-miR-423-5p, hsa-miR-424-5p, hsa-miR-425-3p, hsa-miR-425-5p, hsa-miR-451a, hsa-miR-454-3p, hsa-miR-483-5p, hsa-miR-484, hsa-miR-485-3p, hsa-miR-486-5p, hsa-miR-495-3p, hsa-miR-497-5p, hsa-miR-501-3p, hsa-miR-502-3p, hsa-miR-505-3p, hsa-miR-532-3p, hsa-miR-532-5p, hsa-miR-543, hsa-miR-574-3p, hsa-miR-584-5p, hsa-miR-590-5p, hsa-miR-629-5p, hsa-miR-652-3p, hsa-miR-660-5p, hsa-miR-7-1-3p, hsa-miR-766-3p, hsa-miR-874-3p, hsa-miR-877-5p, hsa-miR-885-5p, hsa-miR-92a-3p, hsa-miR-92b-3p, hsa-miR-93-3p, hsa-miR-93-5p, hsa-miR-99a-5p, hsa-miR-99b-5p.

3. The method according to claim 2, wherein said expression profile is determined of miRNAs selected from the group consisting of: hsa-miR-146a-5p (SEQ ID NO: 45), hsa-miR-126-5p (SEQ ID NO: 25), hsa-miR-425-5p (SEQ ID NO: 147), hsa-miR-145-5p (SEQ ID NO: 44), hsa-miR-25-3p (SEQ ID NO: 95), hsa-miR-221-3p (SEQ ID NO: 86), hsa-miR-374b-5p (SEQ ID NO: 135).

4. The method according to claim 2, wherein said expression profile is determined of miRNAs selected from the group consisting of: hsa-miR-146a-5p (SEQ ID NO: 45), hsa-miR-126-5p (SEQ ID NO: 25), hsa-miR-425-5p (SEQ ID NO: 147), hsa-145-5p (SEQ ID NO: 44), hsa-miR-25-3p (SEQ ID NO: 95).

5. The method according to any one of the claims 1-4, wherein said determined expression profile with respect to said at least one reference expression profile is characterised as follows:
down-regulated miRNA: hsa-miR-146a-5p (SEQ ID NO: 45), and hsa-miR-126-5p (SEQ ID NO: 25).

6. The method according to any one of the claims 1-5, wherein said determined expression profile with respect to said at least one reference expression profile is characterised as follows:
down-regulated miRNA: hsa-miR-146a-5p (SEQ ID NO: 45), hsa-miR-126-5p (SEQ ID NO: 25); hsa-miR-425-5p (SEQ ID NO: 147).

7. The method according to any one of the claims 1-5, wherein said determined expression profile with respect to said at least one reference expression profile is characterised as follows:
down-regulated miRNA: hsa-miR-146a-5p (SEQ ID NO: 45), hsa-miR-126-5p (SEQ ID NO: 25);
up-regulated miRNA: hsa-145-5p (SEQ ID NO: 44), hsa-miR-25-3p (SEQ ID NO: 95).

8. The method according to any one of the claims 1-5, wherein said determined expression profile with respect to said at least one reference expression profile is characterised as follows:
down-regulated miRNA: hsa-miR-146a-5p (SEQ ID NO: 45), hsa-miR-126-5p (SEQ ID NO: 25), hsa-miR-425-5p (SEQ ID NO: 147), has-miR-221-3p (SEQ ID NO: 86);
up-regulated miRNA: hsa-145-5p (SEQ ID NO: 44), hsa-miR-25-3p (SEQ ID NO: 95).

9. The method according to any one of the claims 1-8, wherein said test sample is plasma from venous blood.

10. The method according to any one of the claims 1-9, wherein said subject is an osteoporotic subject having low ASMMI (Appendicular Skeletal Muscle Mass Index), i.e., ASMMI below 5.5 kg/m².


SEQUENCE LISTING (179 sequences)

SEQ ID NO: 1 - RNA, 21 nt, Homo sapiens
  source 1..21: mol_type = other RNA
tgaggtagtaggtgtatagtt

SEQ ID NO: 2 - RNA, 22 nt, Homo sapiens
  source 1..22: mol_type = other RNA
ctatacaacctactgccttccc

SEQ ID NO: 3 - RNA, 22 nt, Homo sapiens
  source 1..22: mol_type = other RNA
tgaggtagtaggttgtgtggtt

SEQ ID NO: 4 - RNA, 22 nt, Homo sapiens
  source 1..22: mol_type = other RNA
tgaggtagtaggttgtatggtt

SEQ ID NO: 5 - RNA, 22 nt, Homo sapiens
  source 1..22: mol_type = other RNA
ctatacgacctgctgcctttct

SEQ ID NO: 6 - RNA, 22 nt, Homo sapiens
  source 1..22: mol_type = other RNA
agaggtagtaggttgcatagtt

SEQ ID NO: 7 - RNA, 22 nt, Homo sapiens
  source 1..22: mol_type = other RNA
tgaggtaggaggttgtatagtt

SEQ ID NO: 8 - RNA, 22 nt, Homo sapiens
  source 1..22: mol_type = other RNA
tgaggtagtagattgtatagtt

SEQ ID NO: 9 - RNA, 22 nt, Homo sapiens
  source 1..22: mol_type = other RNA
tgaggtagtagtttgtacagtt

SEQ ID NO: 10 - RNA, 22 nt, Homo sapiens
  source 1..22: mol_type = other RNA
tgaggtagtagtttgtgctgtt

SEQ ID NO: 11 - RNA, 22 nt, Homo sapiens
  source 1..22: mol_type = other RNA
tggaatgtaaagaagtatgtat

SEQ ID NO: 12 - RNA, 22 nt, Homo sapiens
  source 1..22: mol_type = other RNA
aacccgtagatccgaacttgtg

SEQ ID NO: 13 - RNA, 21 nt, Homo sapiens
  source 1..21: mol_type = other RNA
tacagtactgtgataactgaa

SEQ ID NO: 14 - RNA, 23 nt, Homo sapiens
  source 1..23: mol_type = other RNA
agcagcattgtacagggctatga

SEQ ID NO: 15 - skipped

SEQ ID NO: 16 - RNA, 22 nt, Homo sapiens
  source 1..22: mol_type = other RNA
ccgcactgtgggtacttgctgc

SEQ ID NO: 17 - RNA, 21 nt, Homo sapiens
  source 1..21: mol_type = other RNA
taaagtgctgacagtgcagat

SEQ ID NO: 18 - RNA, 23 nt, Homo sapiens
  source 1..23: mol_type = other RNA
agcagcattgtacagggctatca

SEQ ID NO: 19 - RNA, 23 nt, Homo sapiens
  source 1..23: mol_type = other RNA
taccctgtagaaccgaatttgtg

SEQ ID NO: 20 - RNA, 22 nt, Homo sapiens
  source 1..22: mol_type = other RNA
tggagtgtgacaatggtgtttg

SEQ ID NO: 21 - RNA, 24 nt, Homo sapiens
  source 1..24: mol_type = other RNA
tccctgagaccctttaacctgtga

SEQ ID NO: 22 - RNA, 22 nt, Homo sapiens
  source 1..22: mol_type = other RNA
tccctgagaccctaacttgtga

SEQ ID NO: 23 - RNA, 18 nt, Homo sapiens
  source 1..18: mol_type = other RNA
atcccacctctgccacca

SEQ ID NO: 24 - RNA, 22 nt, Homo sapiens
  source 1..22: mol_type = other RNA
tcgtaccgtgagtaataatgcg

SEQ ID NO: 25 - RNA, 21 nt, Homo sapiens
  source 1..21: mol_type = other RNA
cattattacttttggtacgcg

SEQ ID NO: 26 - RNA, 22 nt, Homo sapiens
  source 1..22: mol_type = other RNA
tcggatccgtctgagcttggct

SEQ ID NO: 27 - RNA, 21 nt, Homo sapiens
  source 1..21: mol_type = other RNA
tcacagtgaaccggtctcttt

SEQ ID NO: 28 - RNA, 22 nt, Homo sapiens
  source 1..22: mol_type = other RNA
cagtgcaatgttaaaagggcat

SEQ ID NO: 29 - RNA, 22 nt, Homo sapiens
  source 1..22: mol_type = other RNA
cagtgcaatgatgaaagggcat

SEQ ID NO: 30 - RNA, 22 nt, Homo sapiens
  source 1..22: mol_type = other RNA
taacagtctacagccatggtcg

SEQ ID NO: 31 - RNA, 22 nt, Homo sapiens
  source 1..22: mol_type = other RNA
tttggtccccttcaaccagctg

SEQ ID NO: 32 - RNA, 22 nt, Homo sapiens
  source 1..22: mol_type = other RNA
tttggtccccttcaaccagcta

SEQ ID NO: 33 - RNA, 22 nt, Homo sapiens
  source 1..22: mol_type = other RNA
catcatcgtctcaaatgagtct

SEQ ID NO: 34 - RNA, 23 nt, Homo sapiens
  source 1..23: mol_type = other RNA
actccatttgttttgatgatgga

SEQ ID NO: 35 - RNA, 23 nt, Homo sapiens
  source 1..23: mol_type = other RNA
tctacagtgcacgtgtctccagt

SEQ ID NO: 36 - RNA, 21 nt, Homo sapiens
  source 1..21: mol_type = other RNA
taccacagggtagaaccacgg

SEQ ID NO: 37 - RNA, 22 nt, Homo sapiens
  source 1..22: mol_type = other RNA
cagtggttttaccctatggtag

SEQ ID NO: 38 - RNA, 22 nt, Homo sapiens
  source 1..22: mol_type = other RNA
taacactgtctggtaaagatgg

SEQ ID NO: 39 - RNA, 23 nt, Homo sapiens
  source 1..23: mol_type = other RNA
tgtagtgtttcctactttatgga

SEQ ID NO: 40 - RNA, 21 nt, Homo sapiens
  source 1..21: mol_type = other RNA
cataaagtagaaagcactact

SEQ ID NO: 41 - RNA, 21 nt, Homo sapiens
  source 1..21: mol_type = other RNA
tgagatgaagcactgtagctc

SEQ ID NO: 42 - RNA, 20 nt, Homo sapiens
  source 1..20: mol_type = other RNA
tacagtatagatgatgtact

SEQ ID NO: 43 - RNA, 22 nt, Homo sapiens
  source 1..22: mol_type = other RNA
ggatatcatcatatactgtaag

SEQ ID NO: 44 - RNA, 23 nt, Homo sapiens
  source 1..23: mol_type = other RNA
gtccagttttcccaggaatccct

SEQ ID NO: 45 - RNA, 22 nt, Homo sapiens
  source 1..22: mol_type = other RNA
tgagaactgaattccatgggtt

SEQ ID NO: 46 - RNA, 22 nt, Homo sapiens
  source 1..22: mol_type = other RNA
tgagaactgaattccataggct

SEQ ID NO: 47 - RNA, 22 nt, Homo sapiens
  source 1..22: mol_type = other RNA
tcagtgcactacagaactttgt

SEQ ID NO: 48 - RNA, 22 nt, Homo sapiens
  source 1..22: mol_type = other RNA
tcagtgcatcacagaactttgt

SEQ ID NO: 49 - RNA, 22 nt, Homo sapiens
  source 1..22: mol_type = other RNA
tctcccaacccttgtaccagtg

SEQ ID NO: 50 - RNA, 21 nt, Homo sapiens
  source 1..21: mol_type = other RNA
ctagactgaagctccttgagg

SEQ ID NO: 51 - RNA, 21 nt, Homo sapiens
  source 1..21: mol_type = other RNA
tcgaggagctcacagtctagt

SEQ ID NO: 52 - RNA, 21 nt, Homo sapiens
  source 1..21: mol_type = other RNA
tcagtgcatgacagaacttgg

SEQ ID NO: 53 - RNA, 22 nt, Homo sapiens
  source 1..22: mol_type = other RNA
taggttatccgtgttgccttcg

SEQ ID NO: 54 - RNA, 23 nt, Homo sapiens
  source 1..23: mol_type = rRNA
ttaatgctaatcgtgataggggt

SEQ ID NO: 55 - RNA, 22 nt, Homo sapiens
  source 1..22: mol_type = rRNA
tagcagcacataatggtttgtg

SEQ ID NO: 56 - RNA, 22 nt, Homo sapiens
  source 1..22: mol_type = rRNA
cgaatcattatttgctgctcta

SEQ ID NO: 57 - RNA, 22 nt, Homo sapiens
  source 1..22: mol_type = rRNA
tagcagcacatcatggtttaca

SEQ ID NO: 58 - RNA, 22 nt, Homo sapiens
  source 1..22: mol_type = rRNA
ccaatattactgtgctgcttta

SEQ ID NO: 59 - RNA, 22 nt, Homo sapiens
  source 1..22: mol_type = rRNA
tagcagcacgtaaatattggcg

SEQ ID NO: 60 - RNA, 23 nt, Homo sapiens
  source 1..23: mol_type = rRNA
caaagtgcttacagtgcaggtag

SEQ ID NO: 61 - RNA, 23 nt, Homo sapiens
  source 1..23: mol_type = other RNA
aacattcaacgctgtcggtgagt

SEQ ID NO: 62 - RNA, 22 nt, Homo sapiens
  source 1..22: mol_type = other RNA
tggagagaaaggcagttcctga

SEQ ID NO: 63 - RNA, 22 nt, Homo sapiens
  source 1..22: mol_type = other RNA
caaagaattctccttttgggct

SEQ ID NO: 64 - RNA, 23 nt, Homo sapiens
  source 1..23: mol_type = other RNA
taaggtgcatctagtgcagatag

SEQ ID NO: 65 - RNA, 23 nt, Homo sapiens
  source 1..23: mol_type = other RNA
taaggtgcatctagtgcagttag

SEQ ID NO: 66 - RNA, 23 nt, Homo sapiens
  source 1..23: mol_type = other RNA
caacggaatcccaaaagcagctg

SEQ ID NO: 67 - RNA, 21 nt, Homo sapiens
  source 1..21: mol_type = other RNA
ctgacctatgaattgacagcc

SEQ ID NO: 68 - RNA, 22 nt, Homo sapiens
  source 1..22: mol_type = other RNA
tgggtctttgcgggcgagatga

SEQ ID NO: 69 - RNA, 22 nt, Homo sapiens
  source 1..22: mol_type = other RNA
tgtaacagcaactccatgtgga

SEQ ID NO: 70 - RNA, 21 nt, Homo sapiens
  source 1..21: mol_type = other RNA
tagcagcacagaaatattggc

SEQ ID NO: 71 - RNA, 22 nt, Homo sapiens
  source 1..22: mol_type = other RNA
ttcaccaccttctccacccagc

SEQ ID NO: 72 - RNA, 22 nt, Homo sapiens
  source 1..22: mol_type = other RNA
acagtagtctgcacattggtta

SEQ ID NO: 73 - RNA, 23 nt, Homo sapiens
  source 1..23: mol_type = other RNA
cccagtgttcagactacctgttc

SEQ ID NO: 74 - RNA, 23 nt, Homo sapiens
  source 1..23: mol_type = other RNA
tgtgcaaatctatgcaaaactga

SEQ ID NO: 75 - RNA, 23 nt, Homo sapiens
  source 1..23: mol_type = other RNA
tgtgcaaatccatgcaaaactga

SEQ ID NO: 76 - RNA, 22 nt, Homo sapiens
  source 1..22: mol_type = other RNA
taacactgtctggtaacgatgt

SEQ ID NO: 77 - RNA, 22 nt, Homo sapiens
  source 1..22: mol_type = other RNA
taacactgtctggtaacgatgt

SEQ ID NO: 78 - RNA, 22 nt, Homo sapiens
  source 1..22: mol_type = other RNA
tccttcattccaccggagtctg

SEQ ID NO: 79 - RNA, 22 nt, Homo sapiens
  source 1..22: mol_type = other RNA
ataagacgagcaaaaagcttgt

SEQ ID NO: 80 - RNA, 23 nt, Homo sapiens
  source 1..23: mol_type = other RNA
taaagtgcttatagtgcaggtag

SEQ ID NO: 81 - RNA, 23 nt, Homo sapiens
  source 1..23: mol_type = other RNA
caaagtgctcatagtgcaggtag

SEQ ID NO: 82 - RNA, 22 nt, Homo sapiens
  source 1..22: mol_type = other RNA
ctgtgcgtgtgacagcggctga

SEQ ID NO: 83 - RNA, 22 nt, Homo sapiens
  source 1..22: mol_type = other RNA
ttggggaaacggccgctgagtg

SEQ ID NO: 84 - RNA, 21 nt, Homo sapiens
  source 1..21: mol_type = other RNA
atgacctatgaattgacagac

SEQ ID NO: 85 - RNA, 22 nt, Homo sapiens
  source 1..22: mol_type = other RNA
tagcttatcagactgatgttga

SEQ ID NO: 86 - RNA, 23 nt, Homo sapiens
  source 1..23: mol_type = other RNA
agctacattgtctgctgggtttc

SEQ ID NO: 87 - RNA, 21 nt, Homo sapiens
  source 1..21: mol_type = other RNA
agctacatctggctactgggt

SEQ ID NO: 88 - RNA, 22 nt, Homo sapiens
  source 1..22: mol_type = other RNA
tgtcagtttgtcaaatacccca

SEQ ID NO: 89 - RNA, 22 nt, Homo sapiens
  source 1..22: mol_type = other RNA
cgtgtatttgacaagctgagtt

SEQ ID NO: 90 - RNA, 22 nt, Homo sapiens
  source 1..22: mol_type = other RNA
aagctgccagttgaagaactgt

SEQ ID NO: 91 - RNA, 22 nt, Homo sapiens
  source 1..22: mol_type = other RNA
agttcttcagtggcaagcttta

SEQ ID NO: 92 - RNA, 21 nt, Homo sapiens
  source 1..21: mol_type = other RNA
atcacattgccagggatttcc

SEQ ID NO: 93 - RNA, 21 nt, Homo sapiens
  source 1..21: mol_type = other RNA
atcacattgccagggattacc

SEQ ID NO: 94 - RNA, 22 nt, Homo sapiens
  source 1..22: mol_type = other RNA
tggctcagttcagcaggaacag

SEQ ID NO: 95 - RNA, 22 nt, Homo sapiens
  source 1..22: mol_type = other RNA
cattgcacttgtctcggtctga

SEQ ID NO: 96 - RNA, 22 nt, Homo sapiens
  source 1..22: mol_type = other RNA
ttcaagtaatccaggataggct

SEQ ID NO: 97 - RNA, 21 nt, Homo sapiens
  source 1..21: mol_type = other RNA
ttcaagtaattcaggataggt

SEQ ID NO: 98 - RNA, 21 nt, Homo sapiens
  source 1..21: mol_type = other RNA
ttcacagtggctaagttccgc

SEQ ID NO: 99 - RNA, 21 nt, Homo sapiens
  source 1..21: mol_type = other RNA
ttcacagtggctaagttctgc

SEQ ID NO: 100 - RNA, 22 nt, Homo sapiens
  source 1..22: mol_type = other RNA
cactagattgtgagctcctgga

SEQ ID NO: 101 - RNA, 22 nt, Homo sapiens
  source 1..22: mol_type = other RNA
aaggagctcacagtctattgag

SEQ ID NO: 102 - RNA, 22 nt, Homo sapiens
  source 1..22: mol_type = other RNA
tagcaccatctgaaatcggtta

SEQ ID NO: 103 - RNA, 23 nt, Homo sapiens
  source 1..23: mol_type = other RNA
tagcaccatttgaaatcagtgtt

SEQ ID NO: 104 - RNA, 22 nt, Homo sapiens
  source 1..22: mol_type = other RNA
tagcaccatttgaaatcggtta

SEQ ID NO: 105 - RNA, 23 nt, Homo sapiens
  source 1..23: mol_type = other RNA
cagtgcaatagtattgtcaaagc

SEQ ID NO: 106 - RNA, 22 nt, Homo sapiens
  source 1..22: mol_type = other RNA
tgtaaacatcctcgactggaag

SEQ ID NO: 107 - RNA, 22 nt, Homo sapiens
  source 1..22: mol_type = other RNA
tgtaaacatcctacactcagct

SEQ ID NO: 108 - RNA, 23 nt, Homo sapiens
  source 1..23: mol_type = other RNA
tgtaaacatcctacactctcagc

SEQ ID NO: 109 - RNA, 22 nt, Homo sapiens
  source 1..22: mol_type = other RNA
tgtaaacatccccgactggaag

SEQ ID NO: 110 - RNA, 22 nt, Homo sapiens
  source 1..22: mol_type = other RNA
ctttcagtcggatgtttacagc

SEQ ID NO: 111 - RNA, 22 nt, Homo sapiens
  source 1..22: mol_type = other RNA
tgtaaacatccttgactggaag

SEQ ID NO: 112 - RNA, 22 nt, Homo sapiens
  source 1..22: mol_type = other RNA
aaaagctgggttgagagggcga

SEQ ID NO: 113 - RNA, 22 nt, Homo sapiens
  source 1..22: mol_type = other RNA
aaaagctgggttgagagggcaa

SEQ ID NO: 114 - RNA, 20 nt, Homo sapiens
  source 1..20: mol_type = other RNA
aaaagctgggttgagagggt

SEQ ID NO: 115 - RNA, 19 nt, Homo sapiens
  source 1..19: mol_type = other RNA
aaaagctgggttgagagga

SEQ ID NO: 116 - RNA, 20 nt, Homo sapiens
  source 1..20: mol_type = other RNA
actgccccaggtgctgctgg

SEQ ID NO: 117 - RNA, 23 nt, Homo sapiens
  source 1..23: mol_type = other RNA
cgcatcccctagggcattggtgt

SEQ ID NO: 118 - RNA, 22 nt, Homo sapiens
  source 1..22: mol_type = other RNA
tattgcacattactaagttgca

SEQ ID NO: 119 - RNA, 20 nt, Homo sapiens
  source 1..20: mol_type = other RNA
cctctgggcccttcctccag

SEQ ID NO: 120 - RNA, 22 nt, Homo sapiens
  source 1..22: mol_type = other RNA
ctggccctctctgcccttccgt

SEQ ID NO: 121 - RNA, 21 nt, Homo sapiens
  source 1..21: mol_type = other RNA
gcccctgggcctatcctagaa

SEQ ID NO: 122 - RNA, 22 nt, Homo sapiens
  source 1..22: mol_type = other RNA
tttttcattattgctcctgacc

SEQ ID NO: 123 - RNA, 23 nt, Homo sapiens
  source 1..23: mol_type = other RNA
tcaagagcaataacgaaaaatgt

SEQ ID NO: 124 - RNA, 22 nt, Homo sapiens
  source 1..22: mol_type = other RNA
tccagcatcagtgattttgttg

SEQ ID NO: 125 - RNA, 23 nt, Homo sapiens
  source 1..23: mol_type = other RNA
tgagcgcctcgacgacagagccg

SEQ ID NO: 126 - RNA, 23 nt, Homo sapiens
  source 1..23: mol_type = other RNA
tccctgtcctccaggagctcacg

SEQ ID NO: 127 - RNA, 21 nt, Homo sapiens
  source 1..21: mol_type = other RNA
gtgcattgtagttgcattgca

SEQ ID NO: 128 - RNA, 23 nt, Homo sapiens
  source 1..23: mol_type = other RNA
tctcacacagaaatcgcacccgt

SEQ ID NO: 129 - RNA, 22 nt, Homo sapiens
  source 1..22: mol_type = other RNA
tggcagtgtcttagctggttgt

SEQ ID NO: 130 - RNA, 22 nt, Homo sapiens
  source 1..22: mol_type = other RNA
ttatcagaatctccaggggtac

SEQ ID NO: 131 - RNA, 22 nt, Homo sapiens
  source 1..22: mol_type = other RNA
aacacacctattcaaggattca

SEQ ID NO: 132 - RNA, 22 nt, Homo sapiens
  source 1..22: mol_type = other RNA
aattgcacggtatccatctgta

SEQ ID NO: 133 - RNA, 22 nt, Homo sapiens
  source 1..22: mol_type = other RNA
taatgcccctaaaaatccttat

SEQ ID NO: 134 - RNA, 22 nt, Homo sapiens
  source 1..22: mol_type = other RNA
ttataatacaacctgataagtg

SEQ ID NO: 135 - RNA, 22 nt, Homo sapiens
  source 1..22: mol_type = other RNA
atataatacaacctgctaagtg

SEQ ID NO: 136 - RNA, 22 nt, Homo sapiens
  source 1..22: mol_type = other RNA
tttgttcgttcggctcgcgtga

SEQ ID NO: 137 - RNA, 21 nt, Homo sapiens
  source 1..21: mol_type = other RNA
atcatagaggaaaatccacgt

SEQ ID NO: 138 - RNA, 21 nt, Homo sapiens
  source 1..21: mol_type = other RNA
aacatagaggaaattccacgt

SEQ ID NO: 139 - RNA, 21 nt, Homo sapiens
  source 1..21: mol_type = other RNA
actggacttggagtcagaagg

SEQ ID NO: 140 - RNA, 22 nt, Homo sapiens
  source 1..22: mol_type = other RNA
gaagttgttcgtggtggattcg

SEQ ID NO: 141 - RNA, 22 nt, Homo sapiens
  source 1..22: mol_type = other RNA
gaatgttgctcggtgaacccct

SEQ ID NO: 142 - RNA, 23 nt, Homo sapiens
  source 1..23: mol_type = other RNA
atcaacagacattaattgggcgc

SEQ ID NO: 143 - RNA, 23 nt, Homo sapiens
  source 1..23: mol_type = other RNA
agctcggtctgaggcccctcagt

SEQ ID NO: 144 - RNA, 23 nt, Homo sapiens
  source 1..23: mol_type = other RNA
tgaggggcagagagcgagacttt

SEQ ID NO: 145 - RNA, 22 nt, Homo sapiens
  source 1..22: mol_type = other RNA
cagcagcaattcatgttttgaa

SEQ ID NO: 146 - RNA, 22 nt, Homo sapiens
  source 1..22: mol_type = other RNA
atcgggaatgtcgtgtccgccc

SEQ ID NO: 147 - RNA, 23 nt, Homo sapiens
  source 1..23: mol_type = other RNA
aatgacacgatcactcccgttga

SEQ ID NO: 148 - RNA, 22 nt, Homo sapiens
  source 1..22: mol_type = other RNA
aaaccgttaccattactgagtt

SEQ ID NO: 149 - RNA, 23 nt, Homo sapiens
  source 1..23: mol_type = other RNA
tagtgcaatattgcttatagggt

SEQ ID NO: 150 - RNA, 22 nt, Homo sapiens
  source 1..22: mol_type = other RNA
aagacgggaggaaagaagggag

SEQ ID NO: 151 - RNA, 22 nt, Homo sapiens
  source 1..22: mol_type = other RNA
tcaggctcagtcccctcccgat

SEQ ID NO: 152 - RNA, 22 nt, Homo sapiens
  source 1..22: mol_type = other RNA
gtcatacacggctctcctctct

SEQ ID NO: 153 - RNA, 22 nt, Homo sapiens
  source 1..22: mol_type = other RNA
tcctgtactgagctgccccgag

SEQ ID NO: 154 - RNA, 22 nt, Homo sapiens
  source 1..22: mol_type = other RNA
aaacaaacatggtgcacttctt

SEQ ID NO: 155 - RNA, 21 nt, Homo sapiens
  source 1..21: mol_type = other RNA
cagcagcacactgtggtttgt

SEQ ID NO: 156 - RNA, 22 nt, Homo sapiens
  source 1..22: mol_type = other RNA
aatgcacccgggcaaggattct

SEQ ID NO: 157 - RNA, 22 nt, Homo sapiens
  source 1..22: mol_type = other RNA
aatgcacctgggcaaggattca

SEQ ID NO: 158 - RNA, 22 nt, Homo sapiens
  source 1..22: mol_type = other RNA
cgtcaacacttgctggtttcct

SEQ ID NO: 159 - RNA, 22 nt, Homo sapiens
  source 1..22: mol_type = other RNA
cctcccacacccaaggcttgca

SEQ ID NO: 160 - RNA, 22 nt, Homo sapiens
  source 1..22: mol_type = other RNA
catgccttgagtgtaggaccgt

SEQ ID NO: 161 - RNA, 22 nt, Homo sapiens
  source 1..22: mol_type = other RNA
aaacattcgcggtgcacttctt

SEQ ID NO: 162 - RNA, 22 nt, Homo sapiens
  source 1..22: mol_type = other RNA
cacgctcatgcacacacccaca

SEQ ID NO: 163 - RNA, 22 nt, Homo sapiens
  source 1..22: mol_type = other RNA
ttatggtttgcctgggactgag

SEQ ID NO: 164 - RNA, 22 nt, Homo sapiens
  source 1..22: mol_type = other RNA
gagcttattcataaaagtgcag

SEQ ID NO: 165 - RNA, 21 nt, Homo sapiens
  source 1..21: mol_type = other RNA
tgggtttacgttgggagaact

SEQ ID NO: 166 - RNA, 21 nt, Homo sapiens
  source 1..21: mol_type = other RNA
aatggcgccactagggttgtg

SEQ ID NO: 167 - RNA, 22 nt, Homo sapiens
  source 1..22: mol_type = other RNA
tacccattgcatatcggagttg

SEQ ID NO: 168 - RNA, 22 nt, Homo sapiens
  source 1..22: mol_type = other RNA
caacaaatcacagtctgccata

SEQ ID NO: 169 - RNA, 23 nt, Homo sapiens
  source 1..23: mol_type = other RNA
tggaagactagtgattttgttgt

SEQ ID NO: 170 - RNA, 22 nt, Homo sapiens
  source 1..22: mol_type = other RNA
actccagccccacagcctcagc

SEQ ID NO: 171 - RNA, 22 nt, Homo sapiens
  source 1..22: mol_type = other RNA
ctgccctggcccgagggaccga

SEQ ID NO: 172 - RNA, 20 nt, Homo sapiens
  source 1..20: mol_type = other RNA
gtagaggagatggcgcaggg

SEQ ID NO: 173 - RNA, 22 nt, Homo sapiens
  source 1..22: mol_type = other RNA
tccattacactaccctgcctct

SEQ ID NO: 174 - RNA, 22 nt, Homo sapiens
  source 1..22: mol_type = other RNA
tattgcacttgtcccggcctgt

SEQ ID NO: 175 - RNA, 22 nt, Homo sapiens
  source 1..22: mol_type = other RNA
tattgcactcgtcccggcctcc

SEQ ID NO: 176 - RNA, 22 nt, Homo sapiens
  source 1..22: mol_type = other RNA
actgctgagctagcacttcccg

SEQ ID NO: 177 - RNA, 23 nt, Homo sapiens
  source 1..23: mol_type = other RNA
caaagtgctgttcgtgcaggtag

SEQ ID NO: 178 - RNA, 22 nt, Homo sapiens
  source 1..22: mol_type = other RNA
aacccgtagatccgatcttgtg

SEQ ID NO: 179 - RNA, 22 nt, Homo sapiens
  source 1..22: mol_type = other RNA
cacccgtagaaccgaccttgcg